(19) United States
(12) Patent Application Publication
(10) Pub. No.: US 2009/0246265 A1
(43) Pub. Date: Oct. 1, 2009

(54) ABUSE DETERRENT TRANSDERMAL FORMULATIONS OF OPIATE AGONISTS AND AGONIST-ANTAGONISTS

(75) Inventors: Audra Lynn Stinchcomb, Lexington, KY (US); Stan Lee Banks, Frankfort, KY (US)

Correspondence Address: MAYER BROWN LLP, P.O. BOX 2828, CHICAGO, IL, 60690, US

(73) Assignee: AllTranz Inc., Lexington, KY (US)

(21) Appl. No.: 12/412,189

(22) Filed: Mar. 26, 2009

Related U.S. Application Data
(60) Provisional application No. 61/039,763, filed on Mar. 26, 2008.

(51) Int. Cl. A61K 9/70; A61P 25/36; A61P 25/04

(52) U.S. Cl. 424/449

(57) ABSTRACT

Described herein are compositions comprising opioids, opioid antagonists and prodrugs of the same, formulations comprising opioids, opioid antagonists and prodrugs of the same, and methods of using opioids, opioid antagonists and prodrugs of the same. One embodiment described herein relates to the transdermal administration of a buprenorphine and encapsulated naltrexone in an abuse-resistant formulation for treating and preventing diseases and/or disorders.

### CROSS REFERENCES TO RELATED APPLICATIONS

This application claims the benefit of U.S. Provisional Application Ser. No. 61/039,763, filed Mar. 26, 2008, which is incorporated herein by reference in its entirety.

### FIELD

Described herein are pharmaceutically active agents suitable for transdermal delivery to a mammal, compositions for transdermal delivery of pharmaceutically active agents and methods of using such compositions in treating and preventing diseases and disorders.

### BACKGROUND

Pain is the most frequently reported symptom and is a common clinical problem confronting the clinician. Millions of people in the United States suffer from severe pain that, according to numerous recent reports, is chronically under-treated or inappropriately managed.

Opioids have long been recognized as one of the most effective treatments of pain. However, they also have a high potential of abuse. In fact, opioid and narcotic abuse are major worldwide problems connected with tremendous social and personal strife. As of 1992, the estimated United States economic cost of drug and alcohol abuse was $246 billion. A recent National Household Survey on Drug Abuse survey conducted by the Substance Abuse and Mental Health Services Administration reported in July 2007 that nearly one in twelve full-time workers in the United States have serious enough drug/alcohol problems to require medical treatment. Providing recovery assistance for drug addicts and alcoholics with pharmacological interventions has proven helpful.

Certain opioids, such as buprenorphine, butorphanol, dezocine, meptazinol, nalbuphine, and pentazocine, have both agonist and antagonist qualities. For example, the main agonist-antagonist effect of buprenorphine is through its binding to μ-opioid and κ-opioid receptors, acting clinically as an agonist at lower doses and as an antagonist at higher doses. The dual agonist-antagonist activity of these opioids make them effective at not only treating pain, but also at reducing the severity of the withdrawal symptoms experienced when a former abuser begins to eliminate opioid and/or alcohol. Buprenorphine is currently available as a sublingual dosage form, both alone (Subutex®) and in combination with naloxone (Suboxone®) for the treatment of pain and opioid dependence. The sublingual administration of these formulations results in clinically relevant drawbacks. For example, the necessity of taking multiple daily doses, or even once-daily dosing, decreases patient compliance. In addition, the daily and multiple daily dosing necessary with sublingual dosage forms may cause more frequent and more extreme peaks and troughs in the blood-plasma concentration of the active medications. These peaks and troughs increase the potential for a patient to experience both the adverse effects associated with supra-therapeutic concentrations and ineffective relief associated with below therapeutic concentrations. Additionally, many sublingual tablets have a bitter taste, which reduces patient compliance.

Further, patients undergoing withdrawal from narcotic or alcohol abuse and those suffering from chronic, under-treated or intractable pain often also suffer from a lack of appetite, nausea and/or frequent emesis. As such, oral and sublingual therapies for these patients are often either poorly tolerated or fail to provide an effective therapeutic dose.

For these patients, transdermal administration can provide a favorable route of administration. Transdermal dosing, provides the patient with a desirable systemic delivery profile which can minimize or eliminate any “highs” (dizziness and drowsiness) associated with more rapid absorption and can reduce the side effects associated with oral administration of a drug such as abdominal pain, nausea and vomiting. Additionally, transdermal administration avoids first-pass metabolism which can allow for higher therapeutic concentrations to be achieved. Transdermal delivery also offers a patient freedom from injections and surgical implantations. Transdermal delivery can also improve patient compliance by reducing the dose frequency. A transdermal patch can offer sustained release of a drug for an extended period (e.g., one week) while transdermal gels are also an accepted dosage form for convenient daily application.

Because of the inherent potential for abuse, it is important that any pharmaceutical composition containing an opioid agonist be made as abuse-resistant or abuse-deterrent as possible. This is particularly true with extended release opioid products, including transdermal applications. Illicit users often will attempt to circumvent the extended release properties of these dosage forms by injecting, chewing or otherwise misusing the product in order to achieve an immediate release of the opioid agonist.

The Food and Drug Administration (“FDA”) has recently emphasized the importance of reducing the risk of opioid abuse. In a Feb. 9, 2009 press release, the FDA publicly announced a program in which it would meet with the manufacturers of extended release and transdermal opioids regarding opioid misuse and abuse. Under the terms of the announced program, the manufactures will be required to develop Risk Evaluation and Mitigations Strategies to ensure proper opioid use.

Thus, it would be desirable to transdermally administer an opioid agonist or antagonist or agonist-antagonist, such as buprenorphine, where the formulation or dosage form used to deliver the opioid agonist or agonist-antagonist is resistant to possible abuse or other illicit diversion.

### SUMMARY

Some embodiments described herein, include an opioid agonist or agonist-antagonist, or a prodrug thereof, in an abuse resistant composition for transdermal delivery of the opioid.

Other embodiments, objects, features and advantages will be set forth in the detailed description of the embodiments that follows, and in part will be apparent from the description, or may be learned by practice, of the claimed invention. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### BRIEF DESCRIPTION OF THE DRAWINGS

FIG. 1 is a side view of a bi-layer patch.

FIG. 2 is a plot of release profiles of gelatin microspheres loaded with naltrexone hydrochloride in water and ethanol.

FIG. 3 is a plot of the cumulative permeation profile of buprenorphine from gel formulation through human skin in vitro.

FIG. 4 is a plot of the cumulative permeation of buprenorphine from adhesive matrix through human skin in vitro.

FIG. 5 is a plot of the cumulative permeation of naltrexone-HCl and a cellulose acetate phthalate coating and naltrexone-HCl complex.

FIG. 6 is a plot of the naltrexone release from a pressure sensitive adhesive patch placed in methanol and ethanol from a cellulose acetate phthalate coating and naltrexone-HCl complex.

### DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

As used herein the terms “abuse resistant” and “abuse deterrent” are synonymous and shall mean any pharmaceutical dosage form or pharmaceutical composition that when misused, prevents the abuser from achieving the non-therapeutic effects sought from misuse of the dosage form or composition, such as opioid induced euphoria.

As used herein an “opioid” refers to compounds that affect opiate receptors, such as the mu, kappa, delta, epsilon, iota, lambda and zeta receptors and includes compounds and substances which activate opiate receptors (“opioid agonists”), inactivate or block opiate receptors (“opioid antagonist”) and partially activate and partially inactivate or block opiate receptors (“opioid agonist-antagonists”). The term opioid also includes natural opiates, semi-synthetic opiates, fully synthetic opioids and endogenous opioid peptides, as well as prodrugs of such compounds. The term opioid also includes any pharmacologically acceptable salts of an opioid.

As used herein a “pH-dependent coating” means a coating, whose solubility is dependent on the pH of solvent.

As used herein “substantially free of an opioid antagonist or prodrug of an opioid antagonist” shall mean that no opioid antagonist or prodrug of an opioid antagonist is separately added to the composition, or the respective element of composition, when the composition is prepared. “Substantially free of an opioid antagonist or prodrug of an opioid antagonist” shall not mean that the no opioid antagonist or prodrug of an opioid antagonist is present in the composition or the respective element of the composition. For example, in one embodiment disclosed herein, a second adhesive matrix layer is substantially free of an opioid antagonist or prodrug of an opioid antagonist as these are not intentionally added to the second adhesive matrix layer. However, the second adhesive matrix layer may, through diffusion or other transport mechanism, contain an amount of the an opioid antagonist or prodrug of an opioid antagonist due to its contact with a first adhesive matrix layer which may contain an opioid antagonist or a prodrug of an opioid antagonist.

As used herein a composition is “substantially free of water” when water has not been separately added to the composition, but may be present in the final composition as a result of the incorporation of other formulation components which contain water and the external absorption of the water from the environment. A composition is “substantially free of water” if water is present in an amount less than about 5% w/w, less than about 2% w/w, less than about 1% w/w, less than about 0.5% w/w or less than about 0.1% w/w of the composition.

As used herein “sub-therapeutic” shall mean an amount which is insufficient to elicit an observable pharmacologic response when administered to a subject.

In one embodiment, the compositions described herein include agonists or agonist-antagonists of opioids. Opioid agonists may be selected from the group comprising alfentanil, allylprodine, alphaprodine, anileridine, benzylmorphine, bezitramide, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydroxypethidine, isomethadone, ketobemidone, levorphanol, levomethadyl, levophenacylmorphan, lofentanil, meperidine, metazocine, methadone, metopon, morphine, myrophine, narceine, nicomorphine, norlevorphanol, normethadone, normorphine, norpipanone, opium, oxycodone, oxymorphone, papavereturn, phenadoxone, phenomorphan, phenazocine, phenoperidine, piminodine, piritramide, propheptazine, promedol, properidine, propoxyphene, sufentanil, tilidine and tramadol.

Opioid agonist-antagonists can be selected from the group comprising buprenorphine, butorphanol, dezocine, meptazinol, nalbuphine, nalorphine and pentazocine. In a further embodiment, the opioid agonist or agonist-antagonist is buprenorphine. In another embodiment, the composition comprises pharmaceutically acceptable prodrugs of opioid agonists or agonist-antagonists. In a further embodiment, the prodrug of the opioid agonist or agonist-antagonist is a pro-drug of buprenorphine.

Due to the potential for opioid agonists and agonist-antagonists drugs to be abused by individuals addicted to opioids, it is desirable to incorporate such compounds into abuse-resistant or abuse-deterrent formulations and dosage forms so that the possibility of abuse through intravenous administration, inhalation, buccal absorption, oral ingestion or other methods of misuse is substantially reduced or eliminated. For example, with transdermal administration, it is desirable to use poorly absorbed forms of opioid antagonists to minimize the effect of the opioid antagonist during transdermal use, but preserving the antagonist properties in the event that abuse of the dosage form is attempted.

In one embodiment, the pharmaceutical composition contains an opioid agonist or agonist-antagonist such as buprenorphine or prodrugs of an opioid agonist or agonist/antagonist, such as a prodrug of buprenorphine and an opioid antagonist. In a further embodiment, the opioid antagonist is selected from the group consisting of: naltrexone (“NTX”), 6-beta-naltrexol, nalbuphine, nalmefene, naloxone, cyclazosine, levallorphan, cyclorphan, oxilorphan and prodrugs of the foregoing. A further embodiment described herein includes a composition in which the naltrexone or a naltrexone prodrug is encapsulated, coated and/or in the form of a microsphere. In a further embodiment, naltrexone or a naltrexone prodrug is encapsulated with a cellulose acetate phthalate coating.

The opioid antagonist particles, created from one of naltrexone, 6-beta-naltrexol, nalmefene, or naloxone, but preferably from naltrexone, 6-beta-naltrexol, or nalmefene, would be insoluble in the dosage form and/or not absorbable at a therapeutic rate through the stratum corneum. These opioid antagonist particles could be created from one or more of the group consisting of poorly absorbed prodrugs, salts, nanoparticles, microparticles, or polymer coatings. Polymer coatings on the opioid antagonist particles would be insoluble in the transdermal dosage form and/or not absorbable at a therapeutic rate across the skin. The polymer coatings could consist of one or more from the group of cellulose acetate phthalate, methacrylate and methyl methacrylate copolymers, methacrylic acid ester copolymers, cellulose, hydroxypropyl methylcellulose, cellulose acetate trimellitate, carboxymethyl ethylcellulose, hydroxypropyl methylcellulose acetate succinate, beeswax, carnauba wax, glyceryl monostearate, stearic acid, palmitic acid, glyceryl monopalmitate, cetyl alcohol, shellac, zein, ethylcellulose, acrylic resins, cellulose acetate, cellulose diacetate, cellulose triacetate, silicone elastomers, calcium carbonate, gum acacia, methylcellulose, hydroxypropylcellulose, polyvinyl alcohol, as well as a variety of known plasticizers and colorants.

In one embodiment, the opioid antagonist would be insoluble in the dosage form and/or not absorbable at a therapeutic rate or extent across the skin. In a further embodiment, when the pharmaceutical composition is used properly, the rate at which the opioid antagonist is absorbed across the skin is insufficient to attenuate the unintended or adverse effects of opioid agonist or agonist-antagonist administration, such as anti-analgesia, hyperalgesia, hyperexcitability, physical dependence, physical tolerance, somnolence and constipation. In a further embodiment, when the pharmaceutical composition is used properly, the rate at which the opioid antagonist is absorbed across the skin is insufficient to enhance the intended pharmacologic effects of the opioid agonist or agonist-antagonist, including the analgesic potency of the opioid agonist or agonist-antagonist.

In a further embodiment, the amount of the opioid antagonist in the pharmaceutical composition is sufficient to block the pharmacological effect of the opioid agonist or agonist-antagonist if the composition is misused. In another embodiment, the amount of the opioid antagonist in the pharmaceutical composition is insufficient to limit the pharmacological activity of the opioid agonist or agonist-antagonist when the dosage form is used properly. In an additional embodiment, the ratio of opioid agonist or agonist-antagonist to opioid antagonist in the pharmaceutical composition is sufficient to block the pharmacological activity of the opioid agonist or agonist-antagonist if the composition is misused, but will not block the pharmacological activity of the opioid agonist or agonist-antagonist when the dosage form is used properly.

In one embodiment, the ratio of opioid agonist or agonist-antagonist to opioid antagonist is about 1 to about 60; 1 to about 50; 1 to about 40; 1 to about 30; 1 to about 20; about 1 to about 10; about 2 to about 10; about 3 to about 10; about 4 to about 10; about 5 to about 10; about 6 to about 10; about 7 to about 10; about 8 to about 10; about 9 to about 10; about 1 to about 9; about 2 to about 9; about 3 to about 9; about 4 to about 9; about 5 to about 9; about 6 to about 9; about 7 to about 9; about 8 to about 9; about 1 to about 8; about 2 to about 8; about 3 to about 8; about 4 to about 8; about 5 to about 8; about 6 to about 8; about 7 to about 8; about 1 to about 7; about 2 to about 7; about 3 to about 7; about 4 to about 7; about 5 to about 7; about 6 to about 7; about 1 to about 6; or about 5 to about 6. In a further embodiment, the ratio of opioid antagonist to opioid agonist or agonist-antagonist is between about 1 to about 60 and about 1 to about 1; about 1 to about 40 and about 1 to about 20; and about 1 to about 15 and about 1 to about 10. In a further embodiment, ratio of opioid antagonist to opioid agonist or agonist-antagonist is about 1 to about 60; 1 to about 50; 1 to about 40; 1 to about 30; 1 to about 20; about 1 to about 10; about 2 to about 10; about 3 to about 10; about 4 to about 10; about 5 to about 10; about 6 to about 10; about 7 to about 10; about 8 to about 10; about 9 to about 10; about 1 to about 9; about 2 to about 9; about 3 to about 9; about 4 to about 9; about 5 to about 9; about 6 to about 9; about 7 to about 9; about 8 to about 9; about 1 to about 8; about 2 to about 8; about 3 to about 8; about 4 to about 8; about 5 to about 8; about 6 to about 8; about 7 to about 8; about 1 to about 7; about 2 to about 7; about 3 to about 7; about 4 to about 7; about 5 to about 7; about 6 to about 7; about 1 to about 6; or about 5 to about 6.

By way of non-limiting illustrative example, the ratio of buprenorphine to naltrexone is about 4:1.

Methods of treating one or more medical conditions such as opioid dependence, alcohol dependence or pain are described herein and comprise administering an opioid agonist or agonist-antagonist or prodrug thereof in an abuse-resistant composition. One embodiment described herein includes a composition of buprenorphine and naltrexone suitable for transdermal administration. Buprenorphine and naltrexone, or prodrugs of either, as described herein may be in any form suitable for administration to a mammal, such as in the form of a free base, free acid, salt, ester, hydrate, anhydrate, enantiomer, isomer, tautomer, polymorph, derivative, or the like, provided that the free base, salt, ester, hydrate, enantiomer, isomer, tautomer, or any other pharmacologically suitable derivative is, or becomes, a therapeutically active form of buprenorphine.

“Pharmaceutically acceptable salts,” or “salts,” include the salts of opioid agonists, agonist-antagonists or antagonists or their respective prodrugs, suitable for administration to a mammal and includes those prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, tosylic, pamoic, napsylic, hydrobromic, valeric, oleic, lauric, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, beta-hydroxybutyric, galactaric and galacturonic acids. The following list of pharmaceutically acceptable salts is not meant to be exhaustive but merely illustrative as person of ordinary skill in the art would appreciate that other pharmaceutically acceptable salts of opioid agonists, agonist-antagonists or antagonists or their respective prodrugs may be prepared.

In one embodiment, acid addition salts can be prepared from the free base forms through a reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. The following list of organic and inorganic acids is not meant to be exhaustive but merely illustrative as person of ordinary skill in the art would appreciate that other acids may be used to create pharmaceutically acceptable salts of opioid agonists, agonist-antagonists or antagonists or their respective prodrugs. In other embodiments, an acid addition salt is reconverted to the free base by treatment with a suitable base. In still other embodiments, the basic salts are alkali metal salts, e.g., sodium salt.

Further embodiments described herein are pharmaceutical compositions comprising (a) buprenorphine; (b) naltrexone; and (c) a pharmaceutical excipient.

Compositions described herein also include those which are suitable for transdermal administration of buprenorphine and naltrexone and optionally include a vehicle or carrier for the transdermal administration of buprenorphine and naltrexone as well as further comprising one or more of the following: pharmacologically active agents, solvents, thickening agents, penetration enhancers, wetting agents, lubricants, emollients, substances added to mask or counteract a disagreeable odor, fragrances, and substances added to improve appearance or texture of the composition as well as other excipients.

Additional embodiments include methods of transdermally administering an opioid agonist or agonist-antagonist and an opioid antagonist or their respective prodrugs to a mammal comprising encapsulating the opioid antagonist and combining the encapsulated opioid antagonist and the opioid agonist or agonist-antagonist with a pharmaceutically acceptable excipient to form a pharmaceutical composition and contacting the pharmaceutical composition with the skin of the mammal to deliver the opioid agonist to the systemic circulation of the mammal, such as a human.

Additional embodiments include methods of transdermally administering buprenorphine and naltrexone or their respective prodrugs to a mammal comprising encapsulating naltrexone and combining the encapsulated naltrexone and buprenorphine with a pharmaceutically acceptable excipient to form a pharmaceutical composition and contacting the pharmaceutical composition with the skin of the mammal to deliver the buprenorphine to the systemic circulation of the mammal, such as a human.

A further embodiment is a method of treating a medical condition in a mammal comprising the steps of administering an opioid agonist or agonist-antagonist and an opioid antagonist or their respective prodrugs wherein the opioid antagonist is encapsulated and combined with the opioid agonist or agonist-antagonist and a pharmaceutically acceptable excipient to form a pharmaceutical composition.

A further embodiment is a method of treating a medical condition in a mammal comprising the steps of administering buprenorphine and naltrexone wherein the naltrexone is encapsulated and combined with buprenorphine and a pharmaceutically acceptable excipient to form a pharmaceutical composition.

In a further embodiment the medical condition is selected from the group consisting of: opioid dependence, alcohol dependence, polydrag addiction and pain.

Combination with Non-Opioid Agents

In one embodiment, the pharmaceutical composition containing the opioid or opioid prodrug could also be combined with an optional second non-opioid pharmacologically active agent for the treatment of pain and/or polydrug abuse, including, for example, a cannabinoid (agonist, antagonist, or inverse agonist), bupropion, hydroxybupropion, nicotine, nornicotine, varenicline, doxepin, acetaminophen, aspirin, or another non-steroidal anti-inflammatory drug. The cannabinoid could consist of one or more of the drugs or prodrugs as described in U.S. patent application Ser. No. 11/157,034, filed Jun. 20, 2005, published as U.S. 2005 0266061 A1 and U.S. patent application Ser. No. 12/182,974, filed Jul. 30, 2008, published as U.S. 2009 036523 A1. The previous listing of suitable compounds for use as an optional second non-opioid pharmacologically active agent is not meant to be exhaustive, as a person of ordinary skill in the art would understand that other compounds (such as those found in the Merck Index, Thirteenth Edition and the Physicians Desk Reference, 58^{th} ed.) would be suitable for use as the optional second non-opioid pharmacologically active agent in the invention disclosed herein. These opioid agonists and/or agonist-antagonists like buprenorphine could also be combined with a second drug for the treatment of pain and/or polydrug abuse, such as a cannabinoid. The cannabinoid could consist of one or more of the following drugs or prodrugs as described in previous patent applications.

Pharmaceutical Excipients

The pharmaceutical compositions described herein can, if desired, include one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a dose unit of the composition. Excipients include, by way of illustration and not limitation, solvents, thickening agents, penetration enhancers, wetting agents, lubricants, emulsifying agents, emollients, substances added to mask or counteract a disagreeable odor, fragrances, antimicrobial preservatives, antioxidants and substances added to improve appearance or texture of the composition. Any such excipients can be used in any dosage forms of the present disclosure. The foregoing list of excipient categories is not meant to be exhaustive but merely illustrative as a person of ordinary skill in the art would recognize that additional excipients could be utilized.

Compositions described herein containing excipients can be prepared by any technique known to a person of ordinary skill in the art of pharmacy, pharmaceutics, drug delivery, pharmacokinetics, medicine or other related discipline that comprises admixing one or more excipients with a therapeutic agent.

In one embodiment, the composition may comprise one or more penetration enhancing agents for transdermal drug delivery. Non-limiting examples of penetration enhancing agents include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate (IPM), butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxy)ethanol (transcutol); diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes. Additional penetration enhancers suitable for use can also be found in U.S. patent application Ser. No. 10/032,163, filed Dec. 21, 2001, published as 2002-0111377 A1.

The penetration enhancing agent is present in an amount sufficient to provide the desired physical properties and skin penetration profile for the composition. Illustratively, one or more pharmaceutically acceptable penetration enhancer can be present in a total amount by weight of the composition of about 0.1%, about 0.2%, about 0.3%, about 0.4%, about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, and or 15.0%. As a further illustration, one or more pharmaceutically acceptable penetration enhancer is present in a total amount by weight between about 0.1% and about 15%; between about 0.1% and about 10%; between about 0.5% and about 10%; or between about 3% and about 8%.

As a further illustration, one or more pharmaceutically acceptable penetration enhancer is present in a total amount by weight between about 1% and about 10%, between about 2% and about 10%, between about 3% and about 10%, between about 4% and about 10%, between about 5% and about 10%, between about 6% and about 10%, between about 7% and about 10%, between about 8% and about 10%, between about 9% and about 10%, between about 1% and about 9%, between about 2% and about 9%, between about 3% and about 9%, between about 4% and about 9%, between about 5% and about 9%, between about 6% and about 9%, between about 7% and about 9%, between about 8% and about 9%, between about 1% and about 8%, between about 2% and about 8%, between about 3% and about 8%, between about 4% and about 8%, between about 5% and about 8%, between about 6% and about 8%, between about 7% and about 8%, between about 1% and about 7%, between about 2% and about 7%, between about 3% and about 7%, between about 4% and about 7%, between about 5% and about 7%, between about 6% and about 7%, between about 1% and about 6%, between about 2% and about 6%, between about 3% and about 6%, between about 4% and about 6%, between about 5% and about 6%, between about 1% and about 5%, between about 2% and about 5%, between about 3% and about 5%, between about 4% and about 5%, between about 1% and about 4%, between about 2% and about 4%, between about 3% and about 4%, between about 1% and about 3%, between about 2% and about 3% and between about 1% and about 2%.

In one embodiment, the composition may comprise a thickening or gelling agent to increase the viscosity of the composition. None-limiting examples of thickening agents (aka gelling agents) which may be used herein include neutralized anionic polymers such as polyacrylic acid (CARBOPOL® by Noveon, Inc., Cleveland, Ohio) (see information at http://www.nuven.com, incorporated by reference herein), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3. Also suitable are other known polymeric thinking agents, such as Pemulen® polymeric emulsifiers, and Noveon® polycarbophils and Klucel®. Additional thickening agents, enhancers and adjuvants may generally be found in Remington's The Science and Practice of Pharmacy as well as the Handbook of Pharmaceutical Excipients, Arthur H. Kibbe ed. 2000. Thickening agents or gelling agents are present in an amount sufficient to provide the desired rheological properties of the composition. Illustratively, one or more pharmaceutically acceptable thickening agent or gelling agent are present in a total amount by weight of about 0.1%, about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75%, about 10%, about 11%, about 11.5%, about 12%, about 12.5%, about 13%, about 13.5%, about 14%, about 14.5% or about 15%. As a further illustration, one or more pharmaceutically acceptable thickening or gelling agent are present in a total amount by weight between about 0.1% and about 15%; about 0.5% and about 5%; or about 1% and about 3%.

In one embodiment a pressure sensitive adhesive is optionally used to assist in affixing a patch containing an opioid to be transdermally delivered to the subject. In a further embodiment, the pressure sensitive adhesive is present in a total amount by weight between about 10% and about 99.9%; about 50% and about 99.9%; about 75% and about 99.9%.

In one embodiment a neutralizing agent is optionally used to assist in forming a gel. Suitable neutralizing agents include sodium hydroxide (e.g., as an aqueous mixture), potassium hydroxide (e.g., as an aqueous mixture), ammonium hydroxide (e.g., as an aqueous mixture), triethanolamine, tromethamine(2-amino 2-hydroxymethyl-1,3 propanediol), aminomethyl propanol (AMP), tetrahydroxypropyl ethylene diamine, diisopropanolamine, Ethomeen C-25 (Armac Industrial Division), Di-2 (ethylhexyl) amine (BASF-Wyandotte Corp., Intermediate Chemicals Division), triamylamine, Jeffamine D-1000 (Jefferson Chemical Co.), b-Dimethylaminopropionitrite (American Cyanamid Co.), Armeen CD (Armac Industrial Division), Alamine 7D (Henkel Corporation), dodecylamine and morpholine. The neutralizing agent is present in an amount sufficient to increase viscosity and form a gel or gel-like composition which is suitable for contact with the skin of a mammal. Illustratively, one or more pharmaceutically acceptable neutralizing agent is present in a total amount by weight of about 0.001%, about 0.0015%, about 0.01%, about 0.015%, about 0.1%, about 0.2%, about 0.3%, about 0.4%, about 0.5%, about 0.6%, about 0.7%, about 0.8%, about 0.9%, about 1.0%, 1.1%, about 1.2%, about 1.3%, about 1.4%, about 1.5%, about 1.6%, about 1.7%, about 1.8%, about 1.9%, about 2.0%, 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%. As a further illustration, one or more pharmaceutically acceptable neutralizing agent is present in a total amount by weight between about 0.1% and about 7% or about 1% and about 5%.

In one embodiment, a solution of sodium hydroxide is used, such as, e.g., 0.01 N, 0.02 N, 0.025 N, 0.05 N, 0.075 N, 0.1 N sodium hydroxide solution, 0.2 N sodium hydroxide solution, 0.5 N sodium hydroxide solution, 1.0 N sodium hydroxide solution, 1.5 N sodium hydroxide solution, 2.0 N sodium hydroxide solution, 10.0 N sodium hydroxide solution, or any other suitable solution for providing an amount sufficient of the aqueous sodium hydroxide to form the desired gel. In one embodiment, the composition results from combining a gelling agent with a neutralizing agent such as about 1% to about 10% (wt/wt) 0.025 N sodium hydroxide, while in another embodiment about 0.1% to about 1% (wt/wt) 0.25 N sodium hydroxide is used. Of course, other suitable neutralizing agents can be used as can other concentrations and amounts of aqueous sodium hydroxide so long as there is a sufficient amount of OH⁻ ions to assist in the formation of a gel.

Compositions described herein optionally comprise one or more pharmaceutically acceptable wetting agents as excipients. Non-limiting examples of surfactants that can be used as wetting agents in compositions of the disclosure include quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10, and octoxynol 9; poloxamers (polyoxyethylene and polyoxypropylene block copolymers); polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether; polyoxyethylene fatty acid esters, for example polyoxyethylene (40) stearate; polyoxyethylene sorbitan esters, for example polysorbate 20 and polysorbate 80 (e.g., Tween™ 80 of ICI); propylene glycol fatty acid esters, for example propylene glycol laurate (e.g., Lauroglycol™ of Gattefossé); sodium lauryl sulfate, fatty acids and salts thereof, for example oleic acid, sodium oleate and triethanolamine oleate; glyceryl fatty acid esters, for example glyceryl monostearate; sorbitan esters, for example sorbitan monolaurate, sorbitan monooleate, sorbitan monopalmitate and sorbitan monostearate; tyloxapol; and mixtures thereof. Such wetting agents, if present, constitute in total about 0.25% to about 15%, about 0.4% to about 10%, or about 0.5% to about 5%, of the total weight of the composition. Illustratively, one or more pharmaceutically acceptable wetting agents are present in a total amount by weight of about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75% or about 10%.

Compositions described herein optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the composition. Illustratively, one or more pharmaceutically acceptable lubricants are present in a total amount by weight of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9% or about 10.0%.

In another embodiment, the compositions described herein optionally comprise an emollient. Illustrative emollients include mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate (IPM), isopropyl palmitate, lecithin, allyl caproate, althea officinalis extract, arachidyl alcohol, argobase EUC, butylene glycol, dicaprylate/dicaprate, acacia, allantoin, carrageenan, cetyl dimethicone, cyclomethicone, diethyl succinate, dihydroabietyl behenate, dioctyl adipate, ethyl laurate, ethyl palmitate, ethyl stearate, isoamyl laurate, octanoate, PEG-75, lanolin, sorbitan laurate, walnut oil, wheat germ oil, super refined almond, super refined sesame, super refined soybean, octyl palmitate, caprylic/capric triglyceride and glyceryl cocoate.

An emollient, if present, is present in the compositions described herein in an amount of about 1% to about 30%, about 3% to about 25%, or about 5% to about 15%, by weight. Illustratively, one or more emollients are present in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In one embodiment, the compositions described herein comprise an antioxidant. Illustrative antioxidants include citric acid, butylated hydroxytoluene (BHT), ascorbic acid, glutathione, retinol, α-tocopherol, β-carotene, α-carotene, ubiquinone, butylated hydroxyanisole, ethylenediaminetetraacetic acid, selenium, zinc, lignan, uric acid, lipoic acid, and N-acetylcysteine. An antioxidant, if present, is present in the compositions described herein in the amount of less than about 1% by weight. Illustratively, one or more antioxidants are present in the total amount of about 0.025%, about 0.05%, about 0.075%, about 0.1%, 0.125%, about 0.15%, about 0.175%, about 0.2%, 0.225%, about 0.25%, about 0.275%, about 0.3%, 0.325%, about 0.35%, about 0.375%, about 0.4%, 0.425%, about 0.45%, about 0.475%, about 0.5%, 0.525%, about 0.55%, about 0.575%, about 0.6%, 0.625%, about 0.65%, about 0.675%, about 0.7%, 0.725%, about 0.75%, about 0.775%, about 0.8%, 0.825%, about 0.85%, about 0.875%, about 0.9%, 0.925%, about 0.95%, about 0.975%, or about 1.0%, by weight. As a further illustration one or more antioxidants are present in the total amount by weight of between about 0.01% and about 1.0%; about 0.05% and about 0.5% or about 0.05% and about 0.2%.

In one embodiment, the compositions described herein comprise an antimicrobial preservative. Illustrative anti-microbial preservatives include acids, including but not limited to benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, or thimerosal. The anti-microbial preservative, if present, is present in an amount of about 0.1% to about 5%, about 0.2% to about 3%, or about 0.3% to about 2%, by weight, for example about 0.2%, about 0.4%, about 0.6%, about 0.8%, about 1%, about 1.2%, about 1.4%, about 1.6%, about 1.8%, about 2%, about 2.2%, about 2.4%, about 2.6%, about 2.8%, about 3.0%, about 3.2%, about 3.4%, about 3.6%, about 3.8%, about 4%, about 4.2%, about 4.4%, about 4.6%, about 4.8%, or about 5%.

Compositions described herein optionally compromise one or more emulsifying agents. The term “emulsifying agent” refers to an agent capable of lowering surface tension between a non-polar and polar phase and includes compounds defined elsewhere as “self emulsifying” agents. Suitable emulsifying agents can come from any class of pharmaceutically acceptable emulsifying agents including carbohydrates, proteins, high molecular weight alcohols, wetting agents, waxes and finely divided solids. The optional emulsifying agent may be present in the composition in a total amount of about 1% to about 25%, about 1% to about 20%, about 1% to about 15%, or about 1% to about 10% by weight of the composition. Illustratively, one or more emulsifying agents are present in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%.

In another embodiment, the water immiscible solvent comprises propylene glycol, and is present in a composition in an amount of about 1% to about 99%, by weight of the composition, for example about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99%.

Composition described herein may optionally comprise one or more alcohols. In a further embodiment, the alcohol is a lower alcohol. As used herein, the term “lower alcohol,” alone or in combination, means a straight-chain or branched-chain alcohol moiety containing one to six carbon atoms. In one embodiment, the lower alcohol contains one to four carbon atoms, and in another embodiment the lower alcohol contains two or three carbon atoms. Examples of such alcohol moieties include ethanol, ethanol USP (i.e., 95% v/v), n-propanol, isopropanol, n-butanol, isobutanol, sec-butanol, and tert-butanol. As used herein, the term “ethanol” refers to C₂H₅OH. It may be used as dehydrated alcohol USP, alcohol USP or in any common form including in combination with various amounts of water. If present, the alcohol is present in an amount sufficient to form a composition which is suitable for contact with a mammal. Illustratively, one or more pharmaceutically acceptable alcohol is present in a total amount by weight of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98%. As a further illustration, one or more pharmaceutically acceptable alcohol is present in a total amount by weight between about 1% and about 98%; between about 10% and about 95%; between about 25% and about 75%; between about 35% and about 70%; or between about 40% and about 50%.

In a further embodiment water is separately added to the composition. The amount of water separately added to a formulation is exclusive of the amount of water independently present in the formulation from any other component (e.g., alcohol, neutralizing agent). Water is present in an amount sufficient to form a composition which is suitable for administration to a mammal. Illustratively, water can be separately added by weight in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% about 91%, about 92%, about 93%, about 94%, about 95%, about 96%, about 97%, or about 98%. As a further illustration, water can be separately added by weight in an amount between about 1% and about 98%; between about 10% and about 70%; between about 10% and about 40%; between about 10% and about 30%; between about 20% and about 30%; or between about 25% and about 30%.

In a further embodiment, the pharmaceutical composition is substantially free of water. In yet a further embodiment, the pharmaceutical composition is anhydrous.

Therapeutic Uses

In another embodiment, compositions described herein which are transdermally administrable include opioid(s) agonists or agonist-antagonists, including buprenorphine, and opioid antagonist(s), including naltrexone.

In another embodiment, compositions described herein which are transdermally administrable include opioid(s) agonists or agonist-antagonists, including buprenorphine, and opioid antagonist(s), including prodrugs of naltrexone.

In another embodiment, compositions described herein which are transdermally administrable include prodrugs of opioid agonists or agonist-antagonists, including prodrugs of buprenorphine, and opioid antagonist prodrugs, including prodrugs of naltrexone.

In another embodiment, compositions described herein which are transdermally administrable include prodrugs of opioid agonists or agonist-antagonists, including prodrugs of buprenorphine, and opioid antagonist prodrugs, including naltrexone.

In another embodiment, compositions disclosed herein comprise one or more opioid agonists or agonist-antagonists, including buprenorphine, in a total amount of about of between about 0.1% and about 95% by weight of the composition. For example, one or more opioid agonists or agonist-antagonists may be present in the amount by weight of: about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90% or about 95%.

In another embodiment, compositions disclosed herein comprise one or more prodrugs of opioid agonists or agonist-antagonists, including prodrugs of buprenorphine, in a total amount of about of between about 0.1% and about 95% by weight of the composition. For example, one or more opioid agonists or agonist-antagonists may be present in the amount by weight of: about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90% or about 95%.

In another embodiment, compositions disclosed herein comprise one or more opioid antagonists, including naltrexone, in a total amount of about of between about 0.1% and about 95% by weight of the composition. For example, one or more opioid antagonists may be present in an amount by weight of: about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90% or about 95%.

In another embodiment, compositions disclosed herein comprise one or more opioid antagonist prodrugs, including prodrugs of naltrexone, in a total amount of about of between about 0.1% and about 95% by weight of the composition. For example, one or more opioid agonists or agonist-antagonists may be present in the amount by weight of: about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, about 50%, about 55%, about 60%, about 65%, about 70%, about 75%, about 80%, about 85%, about 90% or about 95%.

In another embodiment, a single dosage unit comprises a therapeutically effective amount or a therapeutically and/or prophylactically effective amount of buprenorphine or buprenorphine prodrug. The term “therapeutically effective amount” or “therapeutically and/or prophylactically effective amount” as used herein refers to an amount of compound or agent that is sufficient to elicit the required or desired therapeutic and/or prophylactic response, as the particular treatment context may require. Single dosage unit as used herein includes individual patche, sachets containing a single dose, metered pumps designed to dispense a predetermined quantity of material for application to the skin as well as other means for dispensing a single or multiple doses for application to the skin.

The terms “treat”, “treated”, “treating” and “treatment” are to be broadly understood as referring to any response to, or anticipation of, a medical condition in a mammal, particularly a human, and includes but is not limited to:
(i) preventing the medical condition from occurring in a subject, which may or may not be predisposed to the condition, but has not yet been diagnosed with the condition and, accordingly, the treatment constitutes prophylactic treatment for the medical condition;
(ii) inhibiting the medical condition, i.e., arresting, slowing or delaying the on-set, development or progression of the medical condition; or
(iii) relieving the medical condition, i.e., causing regression of the medical condition.

In one embodiment, a therapeutically effective amount of an opioid agonist or agonist-antagonist, such as buprenorphine, is administered transdermally in an abuse-resistant or abuse deterrent formulation to treat a medical condition selected from the group consisting of: opioid dependence, alcohol dependence, polydrug addiction and pain.

In one embodiment, the pharmaceutical composition is administered once daily to a subject in need thereof. In a further embodiment, the pharmaceutical composition comprising an opioid agonist or agonist-antagonist, such as buprenorphine, is administered twice daily to a subject in need thereof. In a further embodiment, the pharmaceutical composition is administered more than twice daily, such as three, four, five, six, seven or eight times daily.

In a further embodiment, the pharmaceutical composition is administered every second day, every third day, every fourth day, every fifth, every sixth day, or once weekly.

Pharmaceutical Dosage Forms

In a further embodiment, the formulation is a gel, an ointment, a cream or a patch and comprises buprenorphine or a buprenorphine prodrug, optionally one or more penetration enhancing agent, thickening agent, lower alcohol, such as ethanol or isopropanol; or water. In another embodiment, the formulation is a gel, an ointment, a cream or a patch, further comprised of sodium hydroxide or triethanolamine or potassium hydroxide, or a combination thereof, in an amount sufficient, as is known in the art, to assist the gelling agent in forming a gel suitable for contact with the skin of a mammal.

The compositions described herein are used in a “pharmacologically effective amount.” This means that the concentration of the drug administered is such that in the composition it results in a therapeutic level of drug delivered over the term that the drug is to be used. Such delivery is dependent on a number of variables including the time period for which the individual dosage unit is to be used, the flux rate of the drug from the composition, for example, buprenorphine or buprenorphine prodrug, from the gel, surface area of application site, etc. For buprenorphine or buprenorphine prodrug, for example, the amount of buprenorphine or buprenorphine prodrug necessary can be experimentally evaluated based on the flux rate of buprenorphine or buprenorphine prodrug through the gel, and through the skin when used with and without enhancers.

In one embodiment, a therapeutically effective dose is between about 1 g and about 10 g, about 2 g and about 8 g, about 3 g and about 7 g, or about 4 g and about 6 g of the composition. In a further embodiment, a therapeutically effective dose is about 1 g, about 1.1 g, about 1.2 g, about 1.3 g, about 1.4 g, about 1.5 g, about 1.6 g, about 1.7 g, about 1.8 g, about 1.9 g, about 2.0 g, about 2.1 g, about 2.2 g, about 2.3 g, about 2.4 g, about 2.5 g, about 2.6 g, about 2.7 g, about 2.8 g, about 2.9 g, about 3.0 g, about 3.1 g, about 3.2 g, about 3.3 g, about 3.4 g, about 3.5 g, about 3.6 g, about 3.7 g, about 3.8 g, about 3.9 g, about 4.0 g, about 4.1 g, about 4.2 g, about 4.3 g, about 4.4 g, about 4.5 g, about 4.6 g, about 4.7 g, about 4.8 g, about 49 g, about 5.0 g, about 5.1 g, about 5.2 g, about 5.3 g, about 5.4 g, about 5.5 g, about 5.6 g, about 5.7 g, about 5.8 g, about 5.9 g, about 6.0 g, about 6.1 g, about 6.2 g, about 6.3 g, about 6.4 g, about 6.5 g, about 6.6 g, about 6.7 g, about 6.8 g, about 6.9 g, about 7.0 g, about 7.1 g, about 7.2 g, about 7.3 g, about 7.4 g, about 7.5 g, about 7.6 g, about 7.7 g, about 7.8 g, about 7.9 g, about 8.0 g, about 8.1 g, about 8.2 g, about 8.3 g, about 8.4 g, about 8.5 g, about 8.6 g, about 8.7 g, about 8.8 g, about 8.9 g, about 9.0 g about 9.1 g, about 9.2 g, about 9.3 g, about 9.4 g, about 9.5 g, about 9.6 g, about 9.7 g, or about 9.8 g, about 9.9 g or about 10 g of the composition.

In one embodiment, compositions described herein are suitable for transdermal administration such as adhesive patches, pastes, gels (e.g., hydroalcoholic, aqueous, etc.), pastes, creams, emulsions, liposomes, ointments, programmable transdermal delivery systems (carbon nanotube based) and other occlusive coverings. In another embodiment, transdermally administrable compositions are adapted for administration in and/or around the abdomen, back, chest, legs, arms, scalp or other suitable skin surface and maybe formulated as patches, ointments, creams, suspensions, lotions, pastes, gels, sprays, foams, oils or other form suitable for transdermal administration.

It will be understood that a therapeutically and/or prophylactically effective amount of a drug for a subject is dependent inter alia on the body weight of the subject as well as other factors known to a person of ordinary skill in the art. A “subject” herein to which a therapeutic agent or composition thereof can be administered includes mammals such as a human subject of either sex and of any age, and also includes any nonhuman animal, particularly a domestic, farm or companion animal, illustratively a cat, cow, pig, dog or a horse as well as laboratory animals such as guinea pigs and primates.

In one embodiment, a single dosage unit of any formulation comprises a therapeutically effective amount or a therapeutically and/or prophylactically effective amount of buprenorphine or a buprenorphine prodrug.

In one embodiment, compositions described herein are suitable for transdermal administration. In another embodiment, transdermally administrable compositions are adapted for administration in and/or around the abdomen, back, chest, legs, arms, scalp or other suitable skin surface and may include formulations in which the buprenorphine is administered in patches, ointments, creams, suspensions, lotions, pastes, gels, sprays, foams or oils.

In one embodiment, a transdermal dosage form, comprising an opioid agonist or an opioid agonist-antagonist and an opioid antagonist, is administered to a subject. In a further embodiment, the transdermal dosage form is a patch which is administered to the subject one time. Following the single administration of the transdermal dosage form, the systemic concentration of the opioid agonist or opioid agonist-antagonist can be measured over time and the maximum concentration (“Cmax”), time to maximum concentration (“Tmax”) and area under the time versus blood plasma or serum concentration curve (“AUC”) can be calculated therefrom. In such an embodiment, AUC can be calculated from 0 to 24 hours or from 0 hours to infinity. In a further embodiment, the transdermal dosage form is administered multiple times to the subject, until the opioid agonist or the opioid antagonist achieves a steady state systemic concentration. After steady has been achieved, the systemic concentration of the opioid agonist or the agonist-antagonist can be measured over time, and a maximum steady state concentration (“Cmax-ss”) and minimum steady state concentration (“Cmin-ss”) of the opioid agonist or the agonist-antagonist can be determined.

In one embodiment, a different transdermal dosage form, comprising the same opioid agonist or an opioid agonist-antagonist and an opioid antagonist of the prior transdermal dosage form, is administered to a subject. In a further embodiment, the different transdermal dosage form satisfies the regulatory requirements for bioequivalence to the prior transdermal dosage form.

In a further embodiment, the different transdermal dosage form is administered to the subject one time. In a further embodiment, the systemic concentration of the opioid agonist or the opioid agonist-antagonist is measured over time and the Cmax, Tmax and AUC resulting from the administration of the different transdermal dosage form is measured. In such an embodiment, AUC can be calculated from 0 to 24 hours or from 0 hours to infinity. In further embodiment, the Cmax, Tmax and AUC of the opioid agonist or the opioid agonist-antagonist from the different transdermal dosage form is between about 60% and 140% of the Cmax, Tmax and AUC of the opioid agonist or the opioid agonist-antagonist from the prior dosage form. In further embodiment, the Cmax, Tmax and AUC of the opioid agonist or the opioid agonist-antagonist from the different transdermal dosage form is between about 80% and 125% of the Cmax, Tmax and AUC of the opioid agonist or the opioid agonist-antagonist from the prior dosage form.

In an additional embodiment, the different transdermal dosage form is administered multiple times to the subject, until the opioid agonist or the opioid antagonist achieves a steady state systemic concentration. In a further embodiment, after steady has been achieved, the systemic concentration of the opioid agonist or the agonist-antagonist from the different transdermal dosage form can be measured over time, and Cmax-ss and Cmin-ss of the opioid agonist or the agonist-antagonist can be determined. In further embodiment, the Cmax-ss and Cmin-ss of the opioid agonist or the opioid agonist-antagonist from the different transdermal dosage form is between about 60% and 140% of the Cmax-ss and Cmin-ss of the opioid agonist or the opioid agonist-antagonist from the prior dosage form. In further embodiment, the Cmax-ss and Cmin-ss of the opioid agonist or the opioid agonist-antagonist from the different transdermal dosage form is between about 80% and 125% of the Cmax-ss and Cmin-ss of the opioid agonist or the opioid agonist-antagonist from the prior dosage form.

Gel Formulations

Alcoholic gels and emulsions have become more popular for systemic delivery of pharmacologically active agents. Testosterone and estradiol products are examples of products on the market now which are gaining market share relative to competitive patch products. Typically patches have been the mainstay for systemic transdermal drug delivery. Ironically, the original transdermal dosage form was a nitroglycerin ointment that was measured out to provide the correct dose. For modern transdermal systemic delivery, many gels and creams have unit dose packaging and calibrated pump dispensers designed to provide the correct dose for application to the skin of the subject. Systemic gel treatments take advantage of the fact that much larger skin surface areas can be covered with the drug, which will improve the chances of therapeutic blood level success. On the other hand, patches are typically associated with certain size restrictions. Alcoholic gels can be made and can optionally include a gelling agent such as ethyl cellulose or a Carbopol. Optionally, appropriate levels of penetration enhancers can be incorporated into the gel.

In another embodiment, the formulation contains an anionic polymer thickening agent precursor such as a carbomer which has been combined with a neutralizer in an amount sufficient to form a gel in the course of forming the composition.

In another embodiment, the formulation contains an anionic polymer thickening agent precursor such as a carbomer which has been combined with a neutralizer in an amount sufficient to form a gel with a viscosity greater than 1000 cps as measured by a Brookfield RV DVII+ Viscometer with a spindle equal to RV6, RPM (rotations per minute) equal to 10, and the temperature maintained at 20° C.

In yet a further embodiment, the formulation contains an anionic polymer thickening agent precursor such as a carbomer which has been combined with a neutralizer selected from the group consisting of sodium hydroxide, ammonium hydroxide, potassium hydroxide, arginine, aminomethyl propanol, tetrahydroxypropyl ethylenediamine, triethanolamine (“TEA”), tromethamine, PEG-15 cocamine, diisopropanolamine, and triisopropanolamine, or combinations thereof in an amount sufficient to neutralize the anionic polymer thickening agent precursor to form a gel in the course of forming the composition. Suitable neutralizing agents and their use with selected anionic polymer thickening agent precursors are disclosed in “Neutralizing Carbopol® and Pemulen® Polymers in Aqueous and Hydroalcoholic Systems,” Commercial Brochure TDS-237 (October 1998) by Noveon Inc. of Cleveland, Ohio, incorporated by reference herein.

In yet a further embodiment, the formulation contains an anionic polymer thickening agent precursor such as a carbomer which has been combined with a neutralizer which is an aqueous solution of sodium hydroxide such as 0.01 N, 0.02 N, 0.025 N, 0.05 N, 0.075 N, 0.1 N sodium hydroxide, or 1.5 N sodium hydroxide, or 2.0 N sodium hydroxide or any other convenient strength aqueous solution in an amount sufficient to form a gel. In one embodiment, the composition was prepared using between about 1.0% and 10.0% 0.025N sodium hydroxide. Accordingly, embodiments employing any percentage between about 1.0% and about 10.0% 0.025 N NaOH may be used, such as, e.g., 1.0%, 2.0%, 3.0%, 4.0%, 5.0%, 6.0%, 7.0%, 8.0%, 9.0% or 10% 0.025 N NaOH.

In an embodiment, the viscosity of the composition of the present invention is about 1,000 cps to about 100,000 cps. Accordingly, the viscosity of the composition of the present invention may be any amount between about 1,000 cps and about 100,000 cps, such as, e.g., about 1,000, about 2,000, about 3,000, about 4,000, about 5,000, about 6,000, about 7,000, about 8,000, about 9,000, about 10,000, about 11,000, about 12,000, about 13,000, about 14,000, about 15,000, about 16,000, about 17,000, about 18,000, about 19,000, about 20,000, about 21,000, about 22,000, about 23,000, about 24,000, about 25,000, about 26,000, about 27,000, about 28,000, about 29,000, about 30,000, about 31,000, about 32,000, about 33,000, about 34,000, about 35,000, about 36,000, about 37,000, about 38,000, about 39,000, about 40,000, about 41,000, about 42,000, about 43,000, about 44,000, about 45,000, about 46,000, about 47,000, about 48,000, about 49,000, about 50,000, about 51,000, about 52,000, about 53,000, about 54,000, about 55,000, about 56,000, about 57,000, about 58,000, about 59,000, about 60,000, about 61,000, about 62,000, about 63,000, about 64,000, about 65,000, about 66,000, about 67,000, about 68,000, about 69,000, about 70,000, about 71,000, about 72,000, about 73,000, about 74,000, about 75,000, about 76,000, about 77,000, about 78,000, about 79,000, about 80,000, about 81,000, about 82,000, about 83,000, about 84,000, about 85,000, about 86,000, about 87,000, about 88,000, about 89,000, about 90,000, about 91,000, about 92,000, about 93,000, about 94,000, about 95,000, about 96,000, about 97,000, about 98,000, about 99,000, about 100,000 cps.

Patch Formulations

The compounds and pharmaceutical compositions described herein are suitable for use in transdermal delivery devices such as patches and the like. For example, the compounds and compositions described herein are suitable for use in a membrane-modulated transdermal delivery system. In this system, the reservoir containing the compound to be transdermally administered to the patient is encapsulated in a shallow compartment molded from a drug impermeable backing and a rate controlling polymeric membrane through which the compound to be delivered passes in a controlled manner. In one embodiment, the external surface of the membrane has a thin layer of a drug-compatible, hypoallergenic adhesive polymer (e.g., silicone or polyacrylate adhesive) which is applied to achieve intimate contact of the transdermal system with the skin.

The compounds and pharmaceutical compositions described herein are also suitable for use in adhesive-diffusion controlled transdermal systems. In these embodiments, the drug reservoir is formulated by directly dispersing the drug (or drugs) to be delivered in an adhesive polymer and then spreading the medicated adhesive onto a flat sheet of drug-impermeable backing membrane or backing layer to form a thin drug reservoir layer. Optionally, on top of the drug reservoir layer, additional layers of non-medicated rate controlling adhesive polymer of constant thickness are placed to produce an adhesive diffusion-controlled drug-delivery system. Also, optionally a second adhesive layer can be added which can contain an active drug substance to be transdermally delivered to the subject.

The compounds and pharmaceutical compositions described herein are also suitable for use in matrix dispersion-type systems. In these systems, the drug reservoir is formed by homogeneously dispersing the drugs in a hydrophilic or lipophilic polymer matrix, and the medicated polymer then is molded into a medicated disc with a defined surface area and controlled thickness. The disc then is glued onto an occlusive baseplate in a compartment fabricated from a drug-impermeable backing. The adhesive polymer is spread along the circumference to form a strip of adhesive rim around the medicated disc.

The compounds and pharmaceutical compositions described herein are also suitable for use in microreservoir systems. In these systems, the drug reservoir is formed by first suspending the drug particles in an aqueous solution of water-soluble polymer and then dispersing it homogeneously in a lipophilic polymer by high-shear mechanical force to form a large number of unleachable, microscopic spheres of drug reservoirs. This unstable dispersion is quickly stabilized by immediately cross-linking, which produces a medicated polymer disc with a constant surface area and fixed thickness. A transdermal therapeutic system is produced in which the medicated disc is positioned at the center and surrounded by an adhesive rim.

Patch formulations can be optimized using in vitro human skin diffusion testing prior to the selection of two or three patches for stability testing. In one embodiment, the drug and adhesive are formulated into one monolithic layer. The drug can be mixed with an adhesive (e.g. silicone type, available from Dow Corning and other manufacturers) in a solvent (e.g. methylene chloride or ethyl acetate). This drug mixture would then be extruded onto a polyester backing film to a uniform thickness of, for example, about 100 microns or greater with a precision wet film applicator. The solvent is allowed to evaporate in a drying oven and the resulting “patch” is trimmed to fit the diffusion cell donor chamber. Various patch formulations will be made until the desired steady-state flux rate and adhesive properties are obtained. Different adhesives can be tried, as well as varying the amount of adhesive in the formulation (Nalluri, Milligan et al. 2005). Suitable results have been obtained by making monolithic patches with DURO-TAK 387-2051, which is an acrylate-vinyl acetate non-curing pressure sensitive adhesive from the National Starch Chemical Company. Different solvents (e.g. isopropyl myristate, propylene glycol) can optionally be incorporated into the formulation in an attempt to optimize the delivery rate. In a further embodiment, reservoir patches can be made if it appears, for example, that the drugs are not compatible with a monolithic matrix patch formulation. In the reservoir system, the active ingredient(s) and any excipient(s) could be formulated into a gel and sealed between a release layer and an impermeable backing material such as polyester or other suitable material known to a person of skill in the art. Ethyl vinyl acetate membranes with acrylic adhesives have been found to be suitable.

Adhesive patch formulations can be prepared containing different loadings of an opioid agonists or agonist-antagonists or prodrugs of the foregoing and an opioid antagonist by using DURO-TAK adhesives (National Starch and Chemical Company, USA). Appropriate amounts of adhesive and drug can be sonicated for ten minutes, cast onto the release liner (9742 Scotchpak, 3M, St. Paul, Minn.) with a wet film applicator (Paul N. Gardner Company, Inc., Pompano Beach, Fla.) set at a 40 mil thickness, and kept at room temperature for one hour and then at 70° C. in an oven for ten minutes (to remove any residual solvent). The patches would then be covered with backing membrane (CoTran 9722, 3M, St. Paul, Minn.), cut into appropriate sizes, and then can be stored in a desiccator for further study.

In further embodiments, additional adhesives which are suitable for preparing patch formulations and transdermal delivery devices such as patches include polyisobutylenes, acrylates, silicone and combinations of the foregoing. Additional adhesives can be found in U.S. patent application Ser. No. 11/907,954, filed Oct. 18, 2007, published as U.S. 2009 017102 A1.

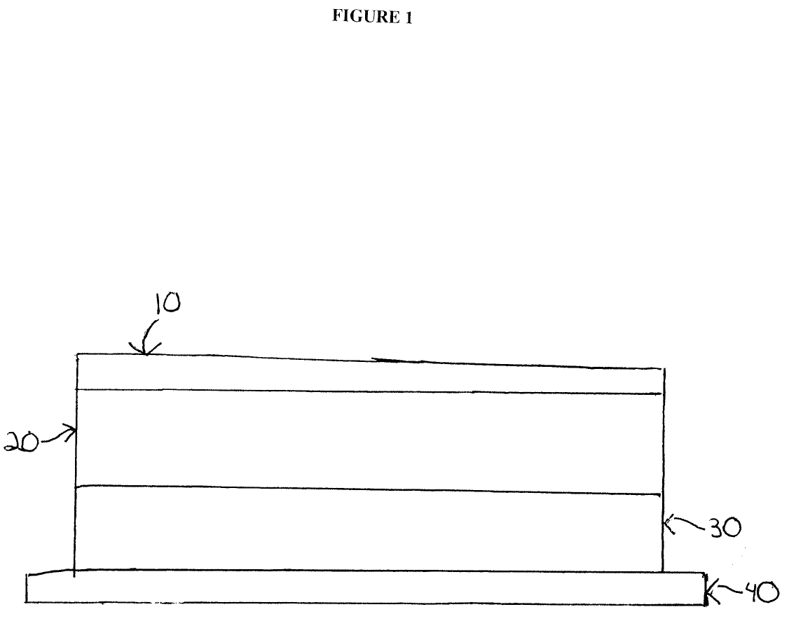

In a further embodiment, the transdermal patch may optionally comprise more than one layer of opioid agonist, opioid agonist-antagonist or opioid antagonist. In a further embodiment, a respective layer may comprise an opioid agonist or an opioid agonist-antagonist alone or in combination with an opioid antagonist. In yet a further embodiment, the opioid antagonist is encapsulated. In an additional embodiment, as set forth in FIG. 1, the transdermal patch comprises two layers of an opioid agonist, opioid an agonist-antagonist or an opioid antagonist, a first layer (20) and a second layer (30). In this embodiment, the first layer (20) is between the second layer (30) and a non-reactive backing layer (10). The non-reactive backing layer (10) may be an occlusive backing, such as Cotran 9715 Film 3M™. Prior to administration to a subject, the second layer (30) is between the first layer (20) and a film covering (40). Illustratively, Scotch Pack 1022 Release Liner 3.0 mil 3M™ may be used as a film covering. When administered to the subject, the film covering (40) is removed and the second layer (30) is placed in direct contact with the subject's skin. In one embodiment, the second layer (30) comprises an opioid agonist or an opioid agonist-antagonist. In a further embodiment, the second layer (30) may optionally also comprise a pressure sensitive adhesive. In an additional embodiment, the first layer (20) comprises an opioid antagonist and either an opioid agonist or an opioid agonist-antagonist. In yet a further embodiment, the opioid antagonist is encapsulated. In a further embodiment, prodrugs of the opioid agonist, opioid agonist-antagonist and opioid antagonist may be used.

In one embodiment, the second (30) has a thickness of between about 0.1 mil and about 100 mil; between about 1 mil and about 50 mil; between about 2 mil and about 20 mil; and between about 5 mil and about 15 mil. Illustratively, the second layer (30) may have a thickness of about 0.1 mil, about 0.2 mil, about 0.3 mil, about 0.4 mil, about 0.5 mil, about 0.6 mil, about 0.7 mil, about 0.8 mil, about 0.9 mil, 1 mil, about 2 mil, about 3 mil, about 4 mil, about 5 mil, about 6 mil, about 7 mil, about 8 mil, about 9 mil, about 10 mil, about 11 mil, about 12 mil, about 13 mil, about 14 mil, about 15 mil, about 16 mil, about 17 mil, about 18 mil, about 19 mil, about 20 mil, about 21 mil, about 22 mil, about 23 mil, about 24 mil, about 25 mil, about 26 mil, about 27 mil, about 28 mil, about 29 mil, about 30 mil, about 31 mil, about 32 mil, about 33 mil, about 34 mil, about 35 mil, about 36 mil, about 37 mil, about 38 mil, about 39 mil, about 40 mil, about 41 mil, about 42 mil, about 43 mil, about 44 mil, about 45 mil, about 46 mil, about 47 mil, about 48 mil, about 49 mil, about 50 mil, about 51 mil, about 52 mil, about 53 mil, about 54 mil, about 55 mil, about 56 mil, about 57 mil, about 58 mil, about 59 mil, about 60 mil, about 61 mil, about 62 mil, about 63 mil, about 64 mil, about 65 mil, about 66 mil, about 67 mil, about 68 mil, about 69 mil, about 70 mil, about 71 mil, about 72 mil, about 73 mil, about 74 mil, about 75 mil, about 76 mil, about 77 mil, about 78 mil, about 79 mil, about 80 mil, about 81 mil, about 82 mil, about 83 mil, about 84 mil, about 85 mil, about 86 mil, about 87 mil, about 88 mil, about 89 mil, about 90 mil, about 91 mil, about 92 mil, about 93 mil, about 94 mil, about 95 mil, about 96 mil, about 97 mil, about 98 mil, about 99 mil or about 100 mil.

In another embodiment, the thickness of the first layer (20) may be increased to achieve longer wear time. In a further embodiment, the first layer (20) has a thickness of between about 0.1 mil and about 100 mil; about 10 mil and about 75 mil; and about 15 mil and about 60 mil. Illustratively, the first layer (20) may have a thickness of about 0.1 mil, about 0.2 mil, about 0.3 mil, about 0.4 mil, about 0.5 mil, about 0.6 mil, about 0.7 mil, about 0.8 mil, about 0.9 mil, 1 mil, about 2 mil, about 3 mil, about 4 mil, about 5 mil, about 6 mil, about 7 mil, about 8 mil, about 9 mil, about 10 mil, about 11 mil, about 12 mil, about 13 mil, about 14 mil, about 15 mil, about 16 mil, about 17 mil, about 18 mil, about 19 mil, about 20 mil, about 21 mil, about 22 mil, about 23 mil, about 24 mil, about 25 mil, about 26 mil, about 27 mil, about 28 mil, about 29 mil, about 30 mil, about 31 mil, about 32 mil, about 33 mil, about 34 mil, about 35 mil, about 36 mil, about 37 mil, about 38 mil, about 39 mil, about 40 mil, about 41 mil, about 42 mil, about 43 mil, about 44 mil, about 45 mil, about 46 mil, about 47 mil, about 48 mil, about 49 mil, about 50 mil, about 51 mil, about 52 mil, about 53 mil, about 54 mil, about 55 mil, about 56 mil, about 57 mil, about 58 mil, about 59 mil, about 60 mil, about 61 mil, about 62 mil, about 63 mil, about 64 mil, about 65 mil, about 66 mil, about 67 mil, about 68 mil, about 69 mil, about 70 mil, about 71 mil, about 72 mil, about 73 mil, about 74 mil, about 75 mil, about 76 mil, about 77 mil, about 78 mil, about 79 mil, about 80 mil, about 81 mil, about 82 mil, about 83 mil, about 84 mil, about 85 mil, about 86 mil, about 87 mil, about 88 mil, about 89 mil, about 90 mil, about 91 mil, about 92 mil, about 93 mil, about 94 mil, about 95 mil, about 96 mil, about 97 mil, about 98 mil, about 99 mil or about 100 mil.

Upon visual examination of the bi-layer system shown in FIG. 1, a potential opioid abuser would be unable to observe a physical distinction between the second layer (30) and first layer (20) after they are placed together. Thus, a potential abuser would not be able to differentiate the second layer (30) containing only the opioid agonist or the opioid agonist-antagonist from the first layer (20) containing both the opioid agonist or the opioid agonist-antagonist and the opioid antagonist.

In another illustrative embodiment, the transdermal patch can be one which is capable of controlling the release of the opioid agonists or agonist-antagonists or prodrugs of the foregoing such that transdermal delivery of the active compound is substantially uniform and sustained over a period of about 6 hours, about 12 hours, about 24 hours, about 48 hours or about 7 days. Such transdermal patch which can be used in the practice of the methods described herein can take the form of an occlusive body having a backing layer. In practice, the occlusive body which includes the opioid agonists or agonist-antagonists or prodrugs of the foregoing is positioned on the subject's skin under conditions suitable for transdermally delivering the active compound to the subject.

Suitable microneedle arrangements for use with the compounds and compositions described herein can be found in the foregoing references as well as in U.S. patent application Ser. No. 11/812,249, filed Jun. 15, 2007, published as U.S. 2008 0008745 A1.

Buprenorphine Prodrugs

The term prodrug as used herein refers to a pharmacologically inert chemical derivative that can be converted, enzymatically or non-enzymatically, in vivo or in vitro, to an active drug molecule, which is capable of exerting one or more physiological effects. As described herein, buprenorphine prodrugs can be used with or instead of buprenorphine and naltrexone prodrugs can be used with or instead of naltrexone.

In one embodiment, illustrative opioid prodrugs include those compounds of Formula (I):

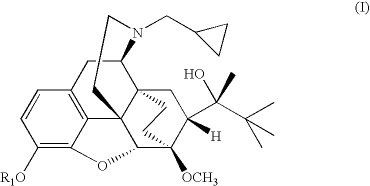

wherein R₁ is comprised of a bio-labile linker (e.g. ester, oxygenated ester, oxaester, pegylated ester, hydroxylated ester, alkyl ester, amino ester, alkylamino ester, dialkylamino ester, carbonate, alkyl carbonate, oxygenated carbonate, pegylated carbonate, hydroxylated carbonate, carbamate, alkyl carbamate, amino carbamate, alkylamino carbamate, dialkylamino carbamate or other suitable bio-labile linking structure) and further comprising moieties which can be selected in order to control the rate and extent of transdermal absorption and metabolism. Several options for R₁ are disclosed herein. Also included herein is the free base, salt, ester, hydrate, amide, enantiomer, isomer, tautomer, polymorph and derivative of compounds of Formula I.

In a further embodiment, the buprenorphine prodrugs can be selected from the group comprising:

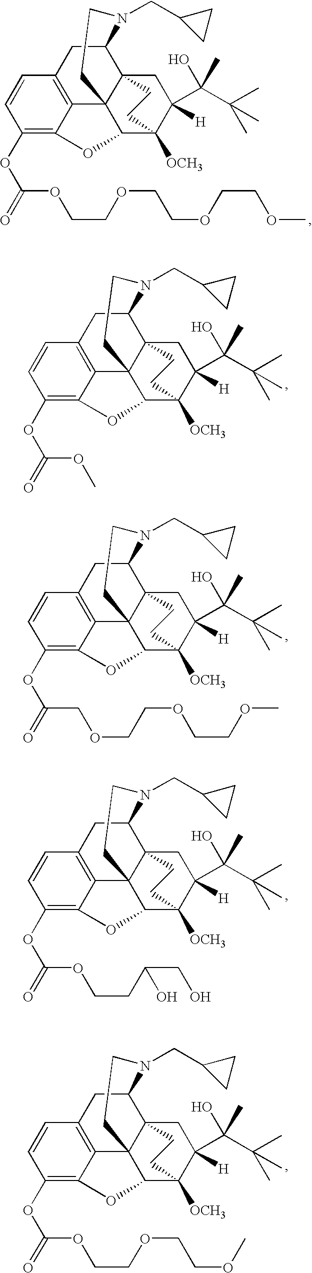

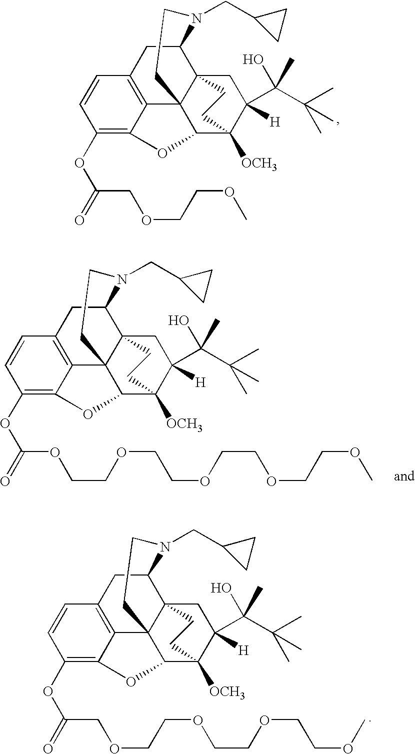

In a further embodiment, one or more buprenorphine prodrugs can be used with or instead of buprenorphine in the pharmaceutical compositions and patches described herein. In an additional embodiment, a buprenorphine prodrug can be used with or instead of buprenorphine in the method of administering buprenorphine to a mammal as described herein. In a further embodiment, a buprenorphine prodrug can be used with or instead of buprenorphine in the method of treating a medical condition by the administration of buprenorphine described herein, wherein the medical conditions is selected from the group consisting of: opioid dependence, polydrug addiction, alcohol dependence and pain.

Naltrexone Prodrugs

In a further embodiment, illustrative opioid antagonist prodrugs include those compounds of Formula (X):

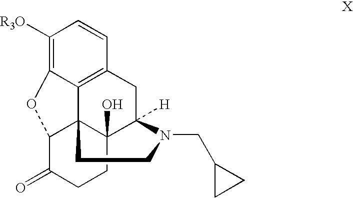

wherein R₃ is comprised of a bio-labile linker (e.g. ester, oxygenated ester, oxaester, pegylated ester, hydroxylated ester, alkyl ester, amino ester, alkylamino ester, dialkylamino ester, carbonate, alkyl carbonate, oxygenated carbonate, pegylated carbonate, hydroxylated carbonate, carbamate, alkyl carbamate, amino carbamate, alkylamino carbamate, dialkylamino carbamate or other suitable bio-labile linking structure) and further comprising moieties which can be selected in order to control the rate and extent of transdermal absorption and metabolism. Several options for R₃ are disclosed herein. Also included herein is the free base, salt, ester, hydrate, amide, enantiomer, isomer, tautomer, polymorph and derivative of compounds of Formula X.

In one embodiment, R₃ is selected from the group consisting of Formula (X), wherein R₃ is selected from the group consisting of:
—COC(CH₃)₃; —COCH(CH₃)₂;
—COCH₂CH(CH₃)₂; —COCH(CH₂CH₃)₂;
—CON(CH₂CH₃)₂, —CON(CH(CH₃)₂)₂;
—COOCH(CH₃)₂;

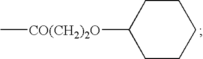

and
—CO(CH₂)₂OCH₃.

In a further embodiment, one or more naltrexone prodrugs can be used with or instead of naltrexone in the pharmaceutical compositions and patches described herein. In an additional embodiment, a naltrexone prodrug can be used with or instead of naltrexone in the method of administering the compositions disclosed herein to a mammal.

Additional embodiments of buprenorphine and naltrexone prodrugs and methods of making buprenorphine and buprenorphine prodrugs contemplated by the present disclosure include, but are not limited to, those described in U.S. patent application Ser. No. 11/860,432, filed Sep. 24, 2007, published as US 2008 0076789 A1 on Mar. 27, 2008.

### EXAMPLES

### Example 1

Naltrexone encapsulation was by the methods described herein. First, 50 mL of paraffin oil was stirred at 1200 RPM. To this solution 20 mL containing 5% w/v gelatin in water and 80 mg of naltrexone hydrochloride was added slowly. The solution was stirred for 15 minutes. Then 30 mL of glyceraldehydes saturated toluene was added and the solution was stirred for 5 h. The mixture was filtered through a 0.45 μm filter, washed twice with acetone, once with isopropanol and finally washed overnight in isopropanol. The microspheres were then dried in an oven at 37° C. the next day.

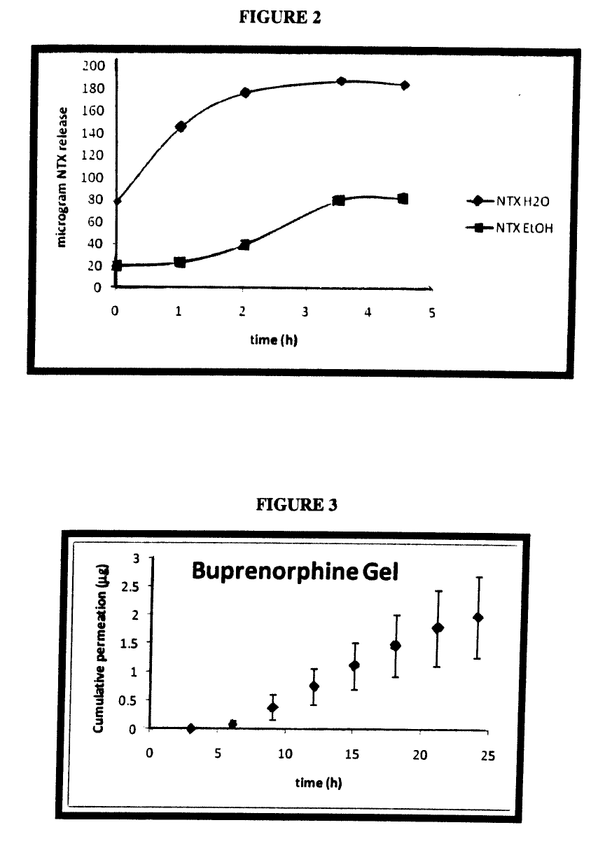

Release studies were performed in methanol and in water to observe release characteristics of naltrexone loaded microspheres. The results of the release studies are shown in FIG. 2. In water the gelatin microspheres showed evidence of burst release which is useful for naltrexone dispersion in the aqueous environment of the plasma or aqueous vehicle in an addict-created injection formula. In ethanol, naltrexone release was characterized by microsphere erosion or controlled release. That is, as naltrexone disperses from the gelatin microspheres, the cavities within the microspheres become more porous allowing more rapid dissolution from the encapsulated naltrexone.

A 1.7% drig in adhesive matrix patch was prepared with DURO-TAK® Elite 87-900A pressure sensitive adhesive and buprenorphine. A 1% naltrexone microsphere solution was added to the matrix and extruded to 30 mil thickness to produce a 1.7% patch.

A 15% cellulose acetate phthalate suspension was prepared in water. The suspension was sonicated for 15 minutes and vortexed for 30 seconds. Next, 1 mL was removed and added to 15 mg of naltrexone hydrochloride. The suspension plus naltrexone was vortexed and sonicated to completely dissolve the readily soluble naltrexone. Finally, the suspension was dried using a Büchi rotovapor R-205° for approximately 1 hour. A dried powder result which could then be easily weighed or stored for further use. The naltrexone cellulose acetate phthalate complex was freely soluble in water. Thus, fast release from the coated naltrexone particles would help to prevent any attempts to abuse the opiate.

A 2% buprenorphine gel was formulated comprising 87% ethanol, 5% propylene glycol and 2% Klucel cellulose polymer and 4% cellulose acetate phthalate coated naltrexone hydrochloride. Release studies of phthalate coated naltrexone were performed by adding approximately 2 mg of coated material to water to test the solubility of the naltrexone complex.

The 1.7% adhesive matrix patches containing microspheres loaded with naltrexone-HCl were adhered to human abdominal dermatomed skin (150 μm thickness). In addition, a 50 μl sample of 2% buprenorphine gel loaded with phthalate coated naltrexone (3.2 mg) was rubbed into the skin and a cap was placed over the cell. Cumulative drug concentration of naltrexone and buprenorphine were monitored for 24 h on a PermeGear flow through diffusion apparatus as described by Paudel et al. (Paudel et al., 2005).

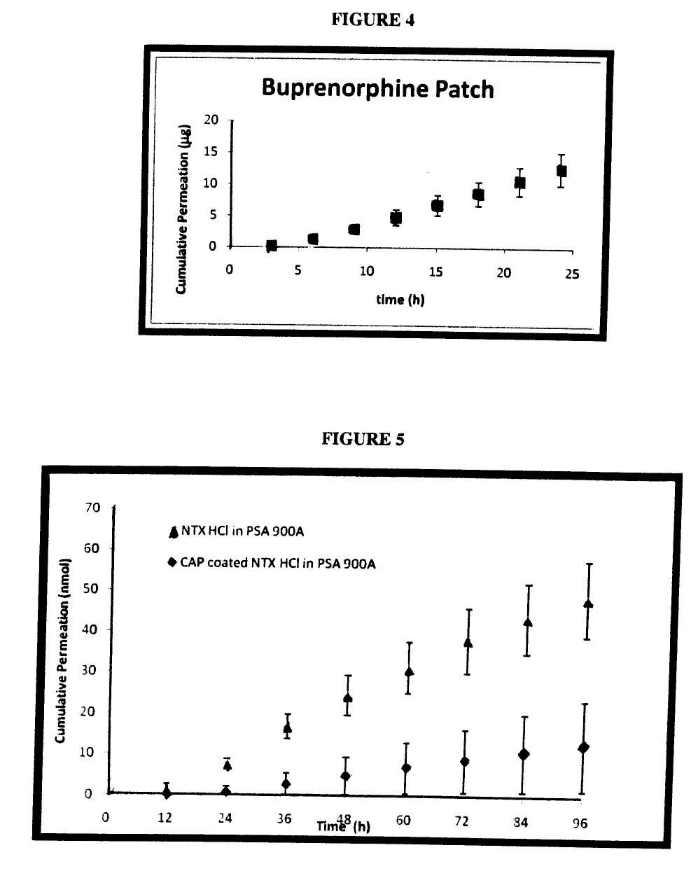

In vitro diffusion studies showed desirable levels of buprenorphine and low flux levels of naltrexone from both the patches and gels. In FIGS. 3 and 4, permeation of buprenorphine through human skin from both a gel formula and drug in adhesive matrix was satisfactory and both maintained steady state levels throughout the 24 hour study. In Table 1, flux values and lag times associated with buprenorphine and naltrexone are listed. These values were estimated from the linear portions of the cumulative permeation profiles.

TABLE 1
Flux and lag time values for buprenorphine and naltrexone
through human skin in vitro

| | Buprenorphine | | Naltrexone | |
|---|---|---|---|---|
| Formu- | Flux | Buprenorphine | Flux | Naltrexone |
| lation | (μg/cm²/h) | Lag time (h) | (μg/cm²/h) | Lag time (h) |
| 2% Gel | 0.13 ± 0.05 | 5.9 ± 1.1 | 0.06 ± 0.02 | 7.6 ± 3.6 |
| 1.7% | 0.7 ± 0.1 | 4.9 ± 0.6 | 0.06 ± 0.02 | 8.8 ± 2.4 |
| Patch | | | | |

Both the formulations deliver a therapeutic rate of buprenorphine. A 20 μg/h delivery rate (comparable to the BuTrans® 20 μg/h system) could be achieved with a 28.6 cm² patch and a 1 g dose of gel spread over 50 cm² could deliver buprenorphine at a therapeutic rate of 6.2 μg/h, similar to the 5 μg/h BuTrans® transdermal system. Naltrexone delivery rates were sub-therapeutic for both the microsphere formula in matrix patch as well as phthalate coated naltrexone-HCl in the gel. The gel was rubbed in leaving the small particles of phthalate coated naltrexone unable to permeate freely through the skin and thus at a slow permeation rate. These levels are unlikely to be therapeutic, as a flux of approximately 12.5 μg/h is required to achieve a therapeutic blockade of opiate receptors.

### Example 2

Two patch formulations for testing were prepared. The patch formulations tested are set forth in Table 2. The patch formulations included naltrexone-HCl and naltrexone-HCl coated with cellulose acetate phthalate (“CAP-NTX”). The NTX-CAP complex was prepared as follows. A 15% suspension of cellulose acetate hydrogen phthalate (acetyl content 17-22%) was prepared in water. To that suspension a 1.5% solution of naltrexone hydrochloride was added. The suspension mixture was shipped to Metrohm USA, where it was dissolved in acetone for spray drying. The solution was spray dried using a GS-310 floor standing laboratory organic spray dryer, resulting in 8.3 g of spray dried product. The coating efficiency as determined by HPLC was 12.2 w/w %.

Durotak Elite 87-900A was used as a pressure sensitive adhesive (“PSA”). In order to prepare the desired 0.5% of naltrexone-HCl drug content in a PSA patch with naltrexone encapsulated with cellulose acetate phthalate, the amount weighed must be adjusted for the coating efficiency. When using a cellulose acetate phthalate coated naltrexone with a coating efficiency of 12.2%, 10 g of Durotak Elite 87-900A should be used with 409.8 mg of NTX-CAP to obtain a 50 mg NTX/10 g PSA. The tested formulations also used Scotch PAK 1022 Release Line 3.0 mil 3M™ as a release liner and Cotran 9715 Film 3M™ as a backing membrane.

TABLE 2
Patch formulations tested

| NTX in PSA | CAP-NTX in PSA |
|---|---|
| 0.5% NTX•HCl | 0.5% NTX•HCl |
| 99.5% pressure sensitive | 99.5% pressure sensitive |
| adhesive | adhesive |
| 3M backing membrane | 3M backing membrane |
| Release Liner | Release Liner |
| Total 100% | 100% |

Neither NTX.HCl nor CAP-NTX were soluble in the pressure sensitive adhesive. Thus, the suspension was mixed and poured to 20 mil thickness.

Permeation and accumulation studies were performed using the methods described herein. Very limited permeation was observed in either patch system. The CAP-NTX complex showed approximately 4 fold less delivery in vitro.

TABLE 3
Permeation data

| Patch | Flux | | |
|---|---|---|---|
| formulation | (nmol/cm²/h) | Lag time (h) | n |
| NTX•HCl in PSA | 0.29 ± 0.06 | 0.0 ± 0.0 | 3 |
| CAP-NTX in PSA | 0.07 ± 0.07 | 11.89 ± 12.09 | 3 |

n = the number of samples tested

Skin concentrations of the naltrexone were low for both formulations, thus little or no depot effect would be observed over 4 days. Both formulations would have very little effect on a transdermal system delivering an opioid agonist. As shown in FIG. 5, a minimal amount of naltrexone accumulates over a 96 hour cumulative profile. Thus, this minimal release from the monolayer of adhesive would have little if any narcotic blockade of an opiate.

TABLE 4
Skin disposition data and cumulative permeation

| Patch | Drug in skin | Cumulative | |
|---|---|---|---|
| formulation | (μmol/g) | (nmol) | n |
| NTX•HCl in PSA | 0.0 ± 0.0 | 48.4 ± 9.3 | 3 |
| CAP-NTX in PSA | 0.76 ± 0.66 | 12.5 ± 11.0 | 3 |

n = the number of samples tested

### Example 3

Four patch formulations for testing were prepared using the methods described in Example 2. The tested patch formulations are set forth in Table 5. The patch formulations included naltrexone-HCl, a pivaloyl ester prodrug of naltrexone, an isopropyl carbonate prodrug of naltrexone and a propyl carbonate prodrug of naltrexone. Durotak Elite 87-900A was used as a PSA. The tested formulations used Scotch PAK 1022 Release Line 3.0 mil 3M™ as a release liner and Cotran 9715 Film 3M™ as a backing membrane.

TABLE 5
Patch Formulations

| NTX•HCl | Pivaloyl ester | Isopropyl carbonate | Propyl carbonate |
|---|---|---|---|
| in PSA | of NTX | of NTX | of NTX |
| 0.5% | 0.5% Pivaloyl | 0.5% Isopropyl | 0.5% Propyl NTX |
| NTX•HCl | NTX | NTX | |
| 99.5% PSA | 99.5% PSA | 99.5% PSA | 99.5% PSA |
| 3M backing | 3M backing | 3M backing | 3M backing |
| membrane | membrane | membrane | membrane |
| Release Liner | Release Liner | Release Liner | Release Liner |
| Total 100% | 100% | 100% | 100% |

Only NTX.HCl was not soluble in the pressure sensitive adhesive. Thus, the suspension and 3 solutions of NTX prodrugs were mixed and poured to 20 mil thickness.

Permeation and accumulation studies were performed using the methods described herein. The total flux was very low for the naltrexone and naltrexone prodrugs tested. This demonstrate that a viable prodrug, coating or water soluble form of naltrexone may be utilized to inhibit abuse of transdermal patches, specifically opiate containing transdermal systems.

TABLE 6
Permeation data

| Patch formulation | Flux (nmol/cm²/h) | Lag time (h) | n |
|---|---|---|---|
| NTX•HCl in PSA | 0.14 ± 0.03 | 7.7 ± 7.1 | 3 |
| Pivaloyl ester of NTX in PSA | 0.19 ± 0.18 | 12.3 ± 15.4 | 3 |
| Propyl carbonate of NTX in | 0.43 ± 0.15 | 12.9 ± 1.2 | 3 |
| PSA | | | |
| Isopropyl carbonate of NTX in | 0.36 ± 0.08 | 10.4 ± 2.6 | 3 |
| PSA | | | |

n = the number of samples tested

Very little permeation was observed in the four formulations tested. Similarly there were low amounts of drug deposited in the skin after 48 h of the patch remaining in contact with the skin. A skin impermeable formulation in a pressure sensitive adhesive to prevent abuse of a potential potent opiate could be prepared from a prodrug of naltrexone.

TABLE 7
Cumulative permeation of NTX•HCl and NTX prodrugs

| | % NTX | Drug in skin | Cumulative | |
|---|---|---|---|---|
| Patch formulation | in skin | (μmol/g) | (nmol) | n |
| NTX•HCl in PSA | 100 | 0.4 ± 0.1 | 9.9 ± 2.3 | 3 |
| Pivaloyl ester of NTX in PSA | 0 | 0.1 ± 0.2 | 10.3 ± 5.6 | 3 |
| Propyl carbonate of NTX in | 50 | 0.3 ± 0.1 | 21.2 ± 4.0 | 3 |
| PSA | | | | |
| Isopropyl carbonate of NTX | 100 | 0.5 ± 0.2 | 23.9 ± 5.1 | 3 |
| in PSA | | | | |

n = the number of samples tested

### Example 4

Three patch formulations for testing were prepared. The tested patch formulations are set forth in Table 8. The patch formulations included naltrexone-HCl, naltrexone-HCl coated with cellulose acetate phthalate and an isobutyryl ester prodrug of naltrexone. The naltrexone-HCl and cellulose acetate phthalate complex was prepared using the methods described in Example 2. The patch formulations also included buprenorphine in a concentration of 2% w/w or 20 mg of buprenorphine per 1 mg of pressure sensitive adhesive. Buprenorphine was freely soluble in the matrix and as well as all prodrugs tested. The naltrexone-HCl and NTX-CAP alone or in patches prepared with buprenorphine were suspensions in the matrix. The PSA admixture was poured onto Scotch PAK 1022 Release Liner 3.0 mil 3M™, spread to an even 20 mil thickness with an 8 path wet film applicator by Gardco®. The matrices were allowed to dry at 40° C. and finally the Cotran 9715 Film 3M™ as a backing membrane was applied.

TABLE 8
Patch formulations of NTX, CAP-NTX and isobutyryl ester of NTX
with 2% buprenorphine w/w % in PSA at 20 mil thickness

| | | Isobutyryl ester of |
|---|---|---|
| NTX•HCl in PAS | NTX-CAP | NTX |
| 0.5% NTX•HCl | 0.5% NTX-CAP | 0.5% Isobutyryl NTX |
| 97.5% PSA | 97.5% PSA | 97.5% PSA |
| 2.0% Buprenorphine | 2.0% Buprenorphine | 2.0% Buprenorphine |
| 3M backing | 3M backing | 3M backing |
| membrane | membrane | membrane |
| Release Liner | Release Liner | Release Liner |
| Total 100% | 100% | 100% |

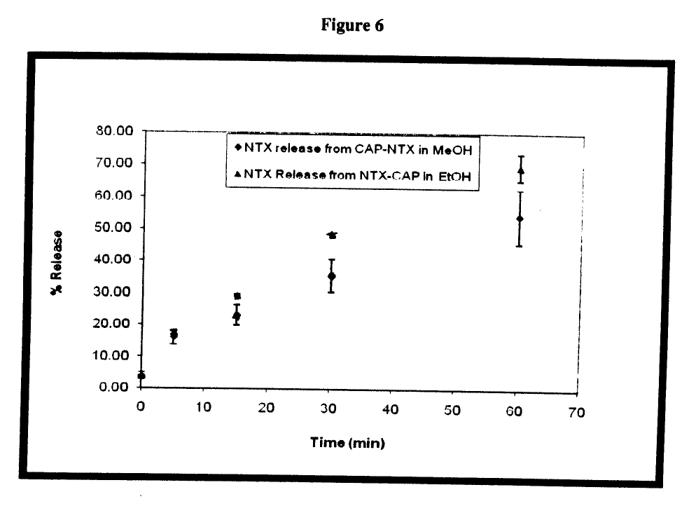

Methanol and ethanol are two media that could potentially be used to dissolve a patch for the purpose of extracting the opiate in the patch by a potential abuse. Thus, an antagonist complex such as NTX-CAP should release in a rapid manner in order to be able to block the opiates effect if injected or ingested. FIG. 6 is a representative profile of the release characteristics of naltrexone coated with cellulose acetate phthalate in methanol and ethanol.

As shown in Tables 9 and 10, the ratio of buprenorphine to naltrexone approaches 4:1. This ratio corresponds to the amount of naltrexone necessary to cause a narcotic blockade at the opiate receptors.

TABLE 9
Release ratio of BUP:NTX from NTX•HCl,
NTX-CAP, or isobutyryl ester of NTX in methanol

| | | | NTX |
|---|---|---|---|
| Time | NTX from | NTX from | from Isobutyryl |
| (min) | NTX•HCl | NTX-CAP | prodrugs of NTX |
| 0 | 7.8 | 4.6 | 4.8 |
| 10 | 6.5 | 3.2 | 5.0 |
| 30 | 6.5 | 2.5 | 5.5 |
| 60 | 6.1 | 2.5 | 5.6 |

TABLE 10
Release ratio of BUP:NTX from NTX•HCl,
NTX-CAP, or isobutyryl ester of NTX in ethanol

| Time | NTX from | NTX from | NTX from Isobutyryl |
|---|---|---|---|
| (min) | NTX•HCl | NTX-CAP | prodrugs of NTX |
| 0 | 9.9 | 8.4 | 4.9 |
| 10 | 7.2 | 4.8 | 4.6 |
| 30 | 4.6 | 3.5 | 4.1 |
| 60 | 4.1 | 3.7 | 4.0 |

### Example 5

The formulations of the patches are set forth in Table 11. Two bi-layer transdermal patches were prepared under the following conditions. The second layer of the patch, which is designed to come in contact with the skin when administered, was prepared by placing 5 g of Durotak Elite 87-900A PSA into a 20 mL plastic vessel. Next, 100 mg of buprenorphine was dissolved in 1.5 mL of ethyl acetate which was added to plastic vessel. The contents were then mixed until dispersed. This layer was extruded onto Scotch PAK 1022 Release Line 3.0 mil 3M™ at a thickness of 10 mil.

The first layer of the patch, which, when administered, is between the second layer and a non-reactive backing layer, was prepared by placing 10 g of Durotak Elite 87-900A PSA into a 20 mL plastic vessel. Next, 200 mg of buprenorphine base dissolved in 1.5 mL ethyl acetate which was added to the plastic vessel and mixed until dispersed. In order to maintain a 1:4 ratio of NTX:BUP, the amount of buprenorphine added to the 10 mil second layer must be taken into account. The amount of naltrexone to be added depends on the form used. Accordingly, if a naltrexone-HCl and buprenorphine formulation is desired, 75 mg of naltrexone-HCl can be dissolved in methanol and added to the dissolved buprenorphine in adhesive. Optionally, if NTX-CAP and buprenorphine patch is desired, 625 mg of NTX-CAP, which was prepared using the methods set-forth in Example 2, can be dispersed directly into the dissolved buprenorphine in adhesive. This suspension turns hazy in color as the insoluble hydrochloride form of naltrexone saturates the solutions or the coated particles of NTX are dispersed. This layer in direct contact was extruded onto Scotch PAK 1022 Release Liner 3.0 mil 3M™ at a thickness of 20 mil.

Both the first layer and the second layer were placed into a drying oven at 40° C. until the solvent system has evaporated. The 20 mil second layer was covered with the non-reactive backing membrane Cotran 9715 Film 3M™. The release liner was pulled away from the 20 mil second layer. The 20 mil second layer was then placed on the 10 mil first layer, opposite the Scotch PAK 1022 Release Line 3.0 mil 3M™. Thereafter, when ready for administration, the Scotch PAK 1022 Release Line 3.0 mil 3M™ is removed from the 10 mil first layer and the patch is applied to the skin of the subject.

TABLE 11
Patch formulations

| NTX•HCl in PSA | NTX-CAP |
|---|---|
| 0.5% NTX•HCl | 0.5% NTX-CAP |
| 97.5% PSA | 97.5% PSA |
| 2.0% Buprenorphine | 2.0% Buprenorphine |
| 3M backing membrane | 3M backing membrane |
| Release Liner | Release Liner |
| Total 100% | 100% |

### Example 6

The formulation of the bi-layer patch is set forth in Table 12. A bi-layer patch formulation, with naltrexone-HCl only, was prepared using the methods described as follows. First, 50 mg naltrexone-HCl was dissolved in 1 mL of methanol. The solution was added drop wise and dispersed in 10 g of a pressure sensitive adhesive. The naltrexone in the methanol solution was not soluble in the pressure sensitive adhesive. Thus, the suspension was extruded to 20 mil thickness. Next 5 g of a pressure sensitive adhesive was extruded to a 10 mil thickness onto a release liner. Both films were dried at 40° C. until dry. A backing membrane was placed on the naltrexone-HCl in adhesive system. The release liner was removed and the adhesive layer with backing membrane was placed on top of the 10 mil thick layer of adhesive only.

TABLE 12
Bi-layer patch formulation with NTX only
NTX•HCl in Bilayer
PSA

0.5% NTX•HCl
99.5% PSA
3M Backing Membrane
Release Liner
Total 100%

Permeation and accumulation studies were performed using the methods as described herein. The studies were done over 24 hours. As shown in Table 13, the total naltrexone flux was low for the naltrexone tested. This suggests that naltrexone could be utilized to inhibit abuse of transdermal patches, specifically opiate containing transdermal systems.

TABLE 13
Permeation Data

| Patch formulation | Flux (nmol/cm²/h) | Lag time (h) | n |
|---|---|---|---|
| NTX•HCl in PSA | 0.27 ± 0.04 | 7.1 ± 2.4 | 3 |

n = the number of samples tested

As shown in Table 14, Only 7.3 nmol of naltrexone passed through the skin. An impermeable formulation in a pressure sensitive adhesive to prevent abuse of a potential potent opiate may be prepared from NTX.HCl.

TABLE 14
Cumulative permeation of NTX•HCl

| | Drug in skin | Cumulative | |
|---|---|---|---|
| Patch formulation | (μmol/g) | (nmol) | n |
| NTX•HCl in PSA | 0.16 ± 0.06 | 7.3 ± 1.1 | 3 |

n = the number of samples tested

### Example 7

The formulations of the bi-layer patches are set forth in Table 15. The bi-layer patches were prepared using the methods previously set-forth in Example 5.

TABLE 15
Bi-layer patch formulations

| NTX•HCl in Bi layer | NTX-CAP in Bi layer |
|---|---|
| PSA | PSA |
| 0.5% NTX•HCl | 0.5% NTX•HCl |
| 2.0% Buprenorphine | 2.0% Buprenorphine |
| 97.5% PSA | 97.5% PSA |
| 3M Backing | 3M Backing |
| Membrane | Membrane |
| Release Liner | Release Liner |
| Total 100% | Total 100% |

As shown in Table 16, the total naltrexone flux was low for the formulations tested and buprenorphine fluxes were 11.2 times higher than naltrexone.

TABLE 16
Permeation data of NTX•HCl and buprenorphine
base in a bi-layer adhesive patch

| NTXHCl and BUP | Flux (nmol/cm²/h) | Lag time (h) | n* |
|---|---|---|---|
| NTX•HCl from bi-layer | 0.02 ± 0.03 | 14.6 ± NA* | 3 |
| BUP from bi-layer | 0.23 ± 0.06 | 10.0 ± 2.6 | 3 |

n = the number of samples tested
*2 out of 3 cells showed no ntx permeation so only one lag time was reported.

As shown in Table 17, small amounts of both compounds buprenorphine and naltrexone were deposited in the skin after 24 h. Only 0.3 nmol of naltrexone passed through the skin. An impermeable bi-layer formulation in a PSA to prevent abuse of a potential potent opiate could be prepared from naltrexone-HCl while allowing the freely permeable buprenorphine to pass through the skin and treat pain locally or systemically.

TABLE 17
Cumulative permeation of NTX•HCl and
buprenorphine base in a bi-layer adhesive patch

| | Drug in skin | Cumulative | |
|---|---|---|---|
| NTX•HCl and BUP | (μmol/g) | (nmol) | n |
| NTX•HCl from bi-layer | 0.29 ± 0.03 | 0.3 ± 0.5 | 3 |
| BUP from bi-layer | 0.35 ± 0.01 | 5.5 ± 0.7 | 3 |

n = the number of samples tested

As shown in Table 18, no naltrexone fluxes were observed while buprenorphine fluxes were observed to pass through the bi-layer and through the skin.

TABLE 18
Permeation data of NTX-CAP and
buprenorphine base in a bi-layer adhesive patch

| | Flux | | |
|---|---|---|---|
| NTX•HCl and BUP | (nmol/cm²/h) | Lag time (h) | n |
| NTX-CAP from bi-layer | 0.0 ± 0.0 | NA | 3 |
| BUP from bi-layer | 0.39 ± 0.05 | 10.6 ± 0.28 | 3 |

n = the number of samples tested

As shown in Table 19, small amounts of both naltrexone and burprenorphine were deposited in the skin after 24 h. While only 0.01 nmol of naltrexone from the NTX-CAP passed through the skin. An impermeable bi-layer formulation in a PSA to prevent abuse of a potential potent opiate could be prepared from NTX-CAP while allowing the freely permeable buprenorphine to pass through the skin and treat pain locally or systemically.

TABLE 19
Cumulative permeation and skin disposition of NTX-CAP
and buprenorphine base in a bi-layer adhesive patch

| | Drug in skin | Cumulative | |
|---|---|---|---|
| NTX-CAP and BUP | (μmol/g) | (nmol) | n |
| NTX-CAP from bi-layer | 0.25 ± 0.03 | 0.01 ± 0.1 | 3 |
| BUP from bi-layer | 0.43 ± 0.01 | 9.3 ± 1.2 | 3 |

n = the number of cells tested

### Example 8

The formulations of the bi-layer patches are set forth in Table 15. The bi-layer patches were prepare using the methods previously set-forth in Example 5.

TABLE 20
Bi-layer Patch Formulation

| NTX•HCl in Bi layer | NTX-CAP in Bi layer |
|---|---|
| PSA | PSA |
| 0.5% NTX•HCl | 0.5% NTX•HCl |
| 2.0% Buprenorphine | 2.0% Buprenorphine |
| 97.5% PSA | 97.5% PSA |
| 3M Backing Membrane | 3M Backing Membrane |
| Release Liner | Release Liner |
| Total 100% | Total 100% |

As shown in Table 21, no naltrexone permeation was observed to calculate a flux or lagtime value as well as standard deviation.

TABLE 22
Permeation data

| NTX•HCl and BUP | Flux (nmol/cm²/h) | Lag time (h) | n |
|---|---|---|---|
| NTX•HCl from bi-layer | 0.0 ± NA* | NA ± NA | 1 |
| BUP from bi-layer | 0.6 ± NA* | 9.5 ± NA | 1 |

n = the number of cells tested

As shown in Table 23, small amounts of both compounds were deposited in the skin after 24 h and only 2.3 nmol of naltrexone passed through the skin and this value was observed at the final time point. An impermeable bi-layer formulation in a PSA to prevent abuse of a potential potent opiate could be prepared from NTX.HCl while allowing the freely permeable buprenorphine to pass through the skin and treat pain locally and systemically.

TABLE 23
Cumulative permeation of NTX•HCl and
buprenorphine base in a bi-layer adhesive patch

| | Drug in skin | Cumulative | |
|---|---|---|---|
| NTX•HCl and BUP | (μmol/g) | (nmol) | n |
| NTX•HCl from bi-layer | 0.30 ± NA | 2.3 ± NA | 1 |
| BUP from bi-layer | 0.37 ± NA | 18.5 ± NA | 1 |

n = the number of cells tested

As shown in Table 24, no naltrexone fluxes were observed while buprenorphine fluxes were observed to pass through the bi-layer and through the skin.

TABLE 24
Permeation data of NTX-CAP and
buprenorphine base in a bi-layer adhesive patch

| | Flux | | |
|---|---|---|---|
| NTX•HCl and BUP | (nmol/cm²/h) | Lag time (h) | n |
| NTX-CAP from bi-layer | 0.0 ± 0.0 | NA | 2 |
| BUP from bi-layer | 1.05 ± 0.65 | 8.2 ± 0.6 | 2 |

n = the number of cells tested

As shown in Table 25, a low amount naltrexone from the NTX-CAP was observed in the skin. Between the two cells, naltrexone was observed in the skin at only 1 time point. Buprenorphine, had a total permeation of 11.65 μg. An impermeable bi-layer formulation in a pressure sensitive adhesive to prevent abuse of a potential potent opiate could be prepared from NTX-CAP while allowing the freely permeable buprenorphine to pass through the skin and treat pain locally or systemically.

TABLE 25
Cumulative permeation and skin disposition of NTX-CAP
and buprenorphine base in a bi-layer adhesive patch

| | Drug in skin | Cumulative | |
|---|---|---|---|
| NTX-CAP and BUP | (μmol/g) | (nmol) | n |
| NTX-CAP from bi- | 0.0 ± 0.0 | 1.5 ± 2.1 | 2 |
| layer | | | |
| BUP from bi-layer | 0.50 ± 0.04 | 24.6 ± 10.8 | 2 |

n = the number of cells tested

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values is stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

We claim:

1. An abuse-resistant transdermal patch for delivering an opioid to a subject, comprising:
(a) a backing layer; and
(b) a first adhesive matrix layer underlying the backing layer, the matrix layer comprising a mixture of:
(i) a therapeutically effective amount of an opioid selected from the group consisting of: an opioid agonist, an opioid agonist prodrug, an opioid agonist-antagonist and an opioid agonist-antagonist prodrug;
(ii) an opioid antagonist or an opioid antagonist prodrug which is:
(A) encapsulated in a coating;
(B) delivered at sub-therapeutic levels to the subject when the patch is used for transdermally administering the opioid agonist or opioid agonist prodrug to the subject; and
(iii) a pressure sensitive adhesive; wherein the first adhesive matrix layer is adapted to be in diffusional communication with the skin of the subject to transdermally administer a therapeutically effective amount of the opioid to the subject.

2. The abuse-resistant transdermal patch of claim 1 further comprising:
(a) a second adhesive matrix layer underlying the first adhesive matrix, the second matrix layer comprising a mixture of:
(i) a therapeutically effective amount of an opioid selected from the group consisting of: an opioid agonist, a prodrug of an opioid agonist, an opioid agonist-antagonist and a prodrug of an opioid agonist-antagonist; and
(ii) a pressure sensitive adhesive; wherein the second adhesive matrix layer is adapted to be in diffusional communication with the skin of the subject to transdermally administer a therapeutically effective amount of the opioid to the subject;
wherein the second adhesive matrix layer is substantially free of an opioid antagonist or prodrug of an opioid antagonist.

3. The abuse-resistant transdermal patch of claim 1, wherein the opioid agonist or opioid agonist prodrug is selected from the group consisting of: alfentanil, allylprodine, alphaprodine, anileridine, benzylmorphine, bezitramide, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydroxypethidine, isomethadone, ketobemidone, levorphanol, levomethadyl, levophenacylmorphan, lofentanil, meperidine, metazocine, methadone, metopon, morphine, myrophine, narceine, nicomorphine, norlevorphanol, normethadone, normorphine, norpipanone, opium, oxycodone, oxymorphone, papavereturn, phenadoxone, phenomorphan, phenazocine, phenoperidine, piminodine, piritramide, propheptazine, promedol, properidine, propoxyphene, sufentanil, tilidine and tramadol and prodrugs of any of the foregoing.

4. The abuse-resistant transdermal patch of claim 1, wherein the opioid agonist-antagonist or opioid agonist-antagonist prodrug is selected from the group consisting of: buprenorphine, butorphanol, dezocine, meptazinol, nalbuphine, nalorphine and pentazocine and prodrugs of any of the foregoing.

5. The abuse-resistant transdermal patch of claim 4, wherein the opioid agonist-antagonist is buprenorphine.

6. The abuse-resistant transdermal patch of claim 4, wherein the opioid agonist-antagonist prodrug is a buprenorphine prodrug.

7. The abuse-resistant transdermal patch of claim 6, wherein the buprenorphine prodrug is selected from the group consisting of:

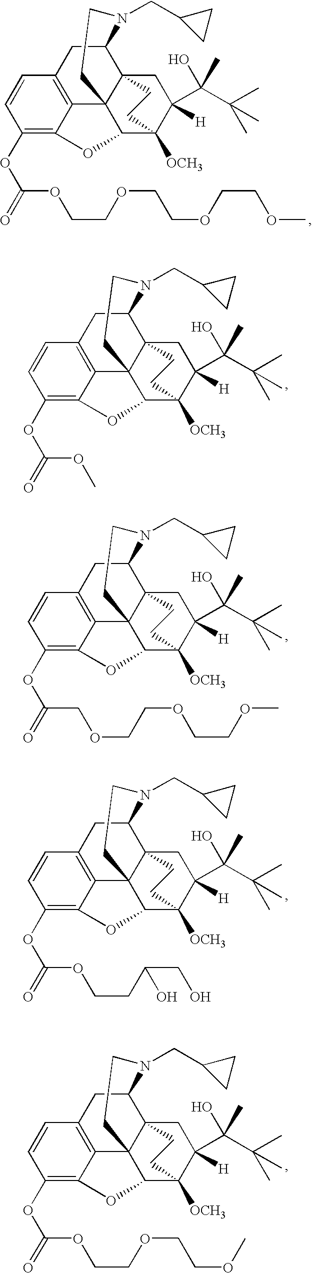

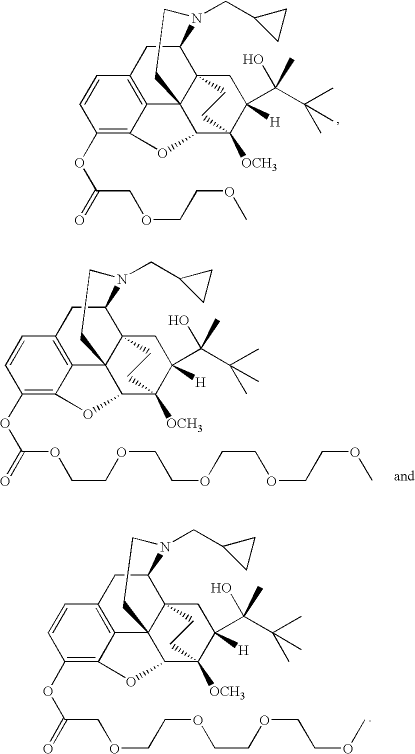

8. The abuse-resistant transdermal patch of claim 1, wherein the opioid antagonist or the opioid antagonist prodrug is selected from the group consisting of: naltrexone, 6-beta-naltrexol, nalbuphine, nalmefene, naloxone, cyclazosine, levallorphan, cyclorphan and oxilorphan and prodrugs of any of the foregoing.

9. The abuse-resistant transdermal patch of claim 8, wherein the opioid antagonist is naltrexone.

10. The abuse-resistant transdermal patch of claim 9, wherein the opioid antagonist prodrug is a naltrexone prodrug.

11. The abuse-resistant transdermal patch of claim 10, wherein the naltrexone prodrug is: 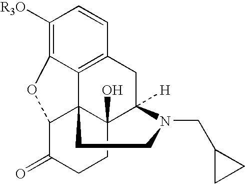
where R₃ is selected from the group consisting of:
—COC(CH₃)₃; —COCH(CH₃)₂;
—COCH₂CH(CH₃)₂; —COCH(CH₂CH₃)₂;
—CON(CH₂CH₃)₂; —CON(CH(CH₃)₂)₂;
—COOCH(CH₃)₂; 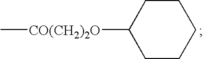
and
—CO(CH₂)₂OCH₃.

12. The abuse-resistant transdermal patch of claim 1, wherein the coating is a pH-dependent coating.

13. The abuse-resistant transdermal patch of claim 12, wherein the pH-dependent coating is cellulose acetate phthalate.

14. The abuse-resistant transdermal patch of claim 1, wherein the first layer is substantially free of water.

15. The abuse-resistant transdermal patch of claim 1, further comprising a penetration enhancer selected from the group consisting of: isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and terpenes.

16. The abuse-resistant transdermal patch of claim 1, wherein the ratio of opioid antagonist to opioid agonist, opioid agonist-antagonist, opioid agonist prodrug or opioid agonist-antagonist prodrug is between about 1:1 and about 1:60.

17. An abuse-resistant pharmaceutical composition for transdermally delivering an opioid, comprising:
(a) a therapeutically effective amount of an opioid selected from the group consisting of: an opioid agonist, a prodrug of an opioid agonist, an opioid agonist-antagonist and a prodrug of an opioid agonist-antagonist; and
(b) an opioid antagonist or prodrug of an opioid antagonist which is:
(i) encapsulated in a coating; and
(ii) delivered at sub-therapeutic levels to the subject when the pharmaceutical composition is used for transdermally administering the opioid to a subject.

18. The abuse-resistant pharmaceutical composition of claim 17, wherein the opioid agonist or the opioid agonist prodrug is selected from the group consisting of: alfentanil, allylprodine, alphaprodine, anileridine, benzylmorphine, bezitramide, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydroxypethidine, isomethadone, ketobemidone, levorphanol, levomethadyl, levophenacylmorphan, lofentanil, meperidine, metazocine, methadone, metopon, morphine, myrophine, narceine, nicomorphine, norlevorphanol, normethadone, normorphine, norpipanone, opium, oxycodone, oxymorphone, papavereturn, phenadoxone, phenomorphan, phenazocine, phenoperidine, piminodine, piritramide, propheptazine, promedol, properidine, propoxyphene, sufentanil, tilidine and tramadol and prodrugs of any of the foregoing.

19. The abuse-resistant pharmaceutical composition of claim 17, wherein the opioid agonist-antagonist or opioid agonist-antagonist prodrug is selected from the group consisting of: buprenorphine, butorphanol, dezocine, meptazinol, nalbuphine, nalorphine and pentazocine and prodrugs of any of the foregoing.

20. The abuse-resistant pharmaceutical composition of claim 17, wherein the opioid antagonist or opioid antagonist prodrug is selected from the group consisting of: naltrexone, 6-beta-naltrexol, nalbuphine, nalmefene, naloxone, cyclazosine, levallorphan, cyclorphan and oxilorphan and prodrugs of any of the foregoing.

21. The abuse-resistant pharmaceutical composition of claim 17, wherein the coating is a pH-dependent coating.

22. The abuse-resistant pharmaceutical composition of claim 21, wherein the pH-dependent coating is cellulose acetate phthalate.

23. The abuse-resistant pharmaceutical composition of claim 17, wherein the first layer is substantially free of water.

24. The abuse-resistant pharmaceutical composition of claim 17, further comprising a penetration enhancer selected from the group consisting of: isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and terpenes.

25. The abuse-resistant pharmaceutical composition of claim 17, wherein the ratio of opioid antagonist to opioid agonist, opioid agonist-antagonist, opioid agonist prodrug or opioid agonist-antagonist prodrug is between about 1:1 and about 1:60.

26. A method of transdermally delivering an opioid to a subject comprising affixing to the skin of the subject the transdermal patch of claim 1 or 2.

27. A method of treating a medical condition comprising affixing to the skin of the subject the transdermal patch of claim 1 or 2; wherein the medical condition is selected from the group consisting of: opioid dependence, alcohol dependence, polydrug addiction and pain.

28. The method of treating a medical condition of claim 27, wherein the opioid agonist or opioid agonist prodrug is selected from the group consisting of: alfentanil, allylprodine, alphaprodine, anileridine, benzylmorphine, bezitramide, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydroxypethidine, isomethadone, ketobemidone, levorphanol, levomethadyl, levophenacylmorphan, lofentanil, meperidine, metazocine, methadone, metopon, morphine, myrophine, narceine, nicomorphine, norlevorphanol, normethadone, normorphine, norpipanone, opium, oxycodone, oxymorphone, papavereturn, phenadoxone, phenomorphan, phenazocine, phenoperidine, piminodine, piritramide, propheptazine, promedol, properidine, propoxyphene, sufentanil, tilidine and tramadol and prodrugs of any of the foregoing.

29. The method of treating a medical condition of claim 27, wherein the opioid agonist-antagonist or opioid agonist-antagonist prodrug selected from the group consisting of: buprenorphine, butorphanol, dezocine, meptazinol, nalbuphine, nalorphine and pentazocine and prodrugs of any of the foregoing.

30. The method of treating a medical condition of claim 27, wherein the opioid antagonist or opioid antagonist prodrug is selected from the group consisting of: naltrexone, 6-beta-naltrexol, nalbuphine, nalmefene, naloxone, cyclazosine, levallorphan, cyclorphan and oxilorphan and prodrugs of any of the foregoing.

31. The method of treating a medical condition of claim 27, wherein the coating is a pH-dependent coating.

32. The method of treating a medical condition of claim 31 wherein the pH-dependent coating is cellulose acetate phthalate.

33. The method of treating a medical condition of claim 27, wherein the first layer is substantially free of water.

34. The method of treating a medical condition of claim 27, further comprising a penetration enhancer.

35. The method of treating a medical condition of claim 27, wherein the ratio of opioid antagonist to opioid agonist, opioid agonist-antagonist, opioid agonist prodrug or opioid agonist-antagonist prodrug is between about 1:1 and about 1:60.

36. An abuse-resistant transdermal patch for delivering an opioid to a subject resulting from the process comprising:
(a) applying a first adhesive matrix layer to a backing layer, the matrix layer comprising a mixture of:
(i) a therapeutically effective amount of an opioid selected from the group consisting of: an opioid agonist, a prodrug of an opioid agonist, an opioid agonist/antagonist and a prodrug of an opioid agonist/antagonist;
(ii) an opioid antagonist or prodrug of an opioid antagonist which is:
(A) encapsulated in a coating;
(B) delivered at sub-therapeutic levels to the subject when the patch is used for transdermally administering the opioid; and
(iii) a pressure sensitive adhesive; wherein the first adhesive matrix layer is adapted to be in diffusional communication with the skin of the subject to transdermally administer a therapeutically effective amount of the opioid to the subject.

37. The abuse-resistant transdermal patch of claim 36, wherein the opioid agonist or opioid agonist prodrug is selected from the group consisting of: alfentanil, allylprodine, alphaprodine, anileridine, benzylmoiphine, bezitramide, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydroxypethidine, isomethadone, ketobemidone, levorphanol, levomethadyl, levophenacylmorphan, lofentanil, meperidine, metazocine, methadone, metopon, morphine, myrophine, narceine, nicomorphine, norlevorphanol, normethadone, normorphine, norpipanone, opium, oxycodone, oxymorphone, papavereturn, phenadoxone, phenomorphan, phenazocine, phenoperidine, piminodine, piritramide, propheptazine, promedol, properidine, propoxyphene, sufentanil, tilidine and tramadol and prodrugs of any of the foregoing.

38. The abuse-resistant transdermal patch of claim 36, wherein the opioid agonist-antagonist or opioid agonist-antagonist prodrug is selected from the group consisting of: buprenorphine, butorphanol, dezocine, meptazinol, nalbuphine, nalorphine and pentazocine and prodrugs of any of the foregoing.

39. The abuse-resistant transdermal patch of claim 36, wherein the opioid antagonist or opioid antagonist prodrug is selected from the group consisting of: naltrexone, 6-beta-naltrexol, nalbuphine, nalmefene, naloxone, cyclazosine, levallorphan, cyclorphan and oxilorphan and prodrugs of any of the foregoing.

40. The abuse-resistant transdermal patch of claim 36, wherein the coating is a pH-dependent coating.

41. The abuse-resistant transdermal patch of claim 40, wherein the pH-dependent coating is cellulose acetate phthalate.

42. The abuse-resistant transdermal patch of claim 36, wherein the first layer is substantially free of water.

43. The abuse-resistant transdermal patch of claim 36, further comprising a penetration enhancer.

44. The abuse-resistant transdermal patch of claim 36, wherein the ratio of opioid antagonist to opioid agonist, opioid agonist-antagonist, opioid agonist prodrug or opioid agonist-antagonist prodrug is between about 1:1 and about 1:60.
